(19) United States
(12) Patent Application Publication
(10) Pub. No.: US 2022/0275416 A1
(43) Pub. Date: Sep. 1, 2022

(54) PERIVASCULAR LYSATES AND USES THEREOF

(71) Applicant: Tissue Regeneration Therapeutics Inc., Toronto (CA)

(72) Inventors: John E. DAVIES, Toronto (CA); Yunqing LI, Toronto (CA)

(21) Appl. No.: 17/632,568

(22) PCT Filed: Aug. 7, 2020
(86) PCT No.: PCT/CA2020/051079

§ 371 Date: Feb. 3, 2022

Related U.S. Application Data
(60) Provisional application No. 62/884,637, filed on Aug. 8, 2019.

(51) Int. Cl. C12P 21/00; C12N 5/073; A61K 35/51; A61K 38/17; A61K 8/98; A61P 29/00; A61P 37/06; A61P 17/00; A61P 17/06; A61Q 19/00

(52) U.S. Cl. CPC C12P 21/00; C12N 5/0605; A61K 35/51; A61K 38/1709; A61K 8/981; A61P 29/00; A61P 37/06; A61P 17/00; A61P 17/06; A61Q 19/00

(57) ABSTRACT

The present disclosure provides methods of preparing perivascular tissue lysates and methods of preparing mesenchymal stem cell (e.g., perivascular stem cell) lysates and total protein products from cultured cells (e.g., cultured perivascular stromal cells). The disclosure also features compositions containing such lysates and total protein products and methods of using the lysates in skincare applications, dermatological applications, cell culture applications, and to treat autoimmune or inflammatory diseases or conditions, local inflammation, and angiogenesis-related diseases or conditions.

### BACKGROUND

Stem cell-based therapies have emerged as potential treatments for a variety of diseases and conditions, such as neurological conditions, inflammatory conditions, weight loss, and cancer. Studies evaluating such therapeutic approaches have typically used one of three treatment methodologies: 1) administration of intact stem cells, 2) administration of medium containing factors secreted by stem cells, or 3) administration of stem cell lysates. While these approaches have shown promise, they are limited in that they do not allow for treatment with the entire complement of proteins produced by stem cells, as the methodologies favor either secreted proteins or intracellular and transmembrane proteins.

Accordingly, there exists a need for improved stem cell compositions that contain all three types of proteins: intracellular, transmembrane, and secreted stem cell proteins.

### SUMMARY OF THE INVENTION

The present invention provides compositions containing mesenchymal stem cell (MSC) proteins, such as human umbilical cord perivascular cell (HUCPVC) total protein products (HTPPs) that contain intracellular, transmembrane, and secreted HUCPVC proteins and methods of making and using such compositions. The compositions can be incorporated into pharmaceutical compositions, cosmetic compositions, and dermatologic compositions to treat autoimmune or inflammatory diseases or conditions, inflammation-related skin diseases and conditions, diseases or conditions characterized by local inflammation, skincare concerns, dermatological indications, and diseases or conditions characterized by a lack of angiogenesis or vascularization. The compositions can also be applied to cultured cells, such as cultured umbilical cord perivascular cells, to improve the health, proliferation, and longevity of the cells in culture.

In a first aspect, featured is a method of producing a human umbilical cord perivascular cell (HUCPVC) total protein product (HTPP), the method including the steps of a) obtaining HUCPVCs;

b) incubating the HUCPVCs in saline at about 25° C. to about 37° C. for about 15 minutes to about 24 hours; c) forming a homogenate from the HUCPVCs of step b); and d) filtering said homogenate to produce the HTPP.

In some embodiments, the HUCPVCs of step a) are cultured HUCPVCs.

In some embodiments, the method further includes, prior to step b), passaging the HUCPVCs in culture from 2 to 10 times. In some embodiments, the cultured HUCPVCs are passaged 3 times.

In some embodiments, the HUCPVCs of step a) are cryopreserved HUCPVCs. In some embodiments, the cryopreserved HUCPVCs were cultured prior to cryopreservation. In some embodiments, the method further includes thawing the cryopreserved HUCPVCs prior to step b).

In some embodiments, the HUCPVCs of step a) are attached to an umbilical cord blood vessel. In some embodiments, the umbilical cord blood vessel is a fresh blood vessel. In some embodiments, the umbilical cord blood vessel is a cryopreserved blood vessel. In some embodiments, step a) further includes the step of detaching the HUCPVCs from the blood vessel, and, optionally, centrifuging the detached HUCPVCs, resuspending the HUCPVCs, and counting the number of HUCPVCs prior to step b).

In some embodiments, step b) includes incubating the HUCPVCs in saline at a volume of about 500,000 cells per mL to about two million cells per mL. In some embodiments, step b) includes incubating the HUCPVCs in saline at a volume of about one million cells per mL.

In some embodiments, step b) includes incubating the HUCPVCs in saline for about 6 hours.

In some embodiments, step b) includes incubating the HUCPVCs at about 37° C.

In some embodiments, the method includes adding saline to the homogenate formed in step c) to reach a concentration of about one million cells per mL.

In some embodiments, the filtering is performed using a filter of about 0.2 μM to about 0.45 μM. In some embodiments, the filtering is performed using a filter of about 0.22 μM.

In some embodiments, the method further includes aliquoting the HTPP after filtration.

In some embodiments, the method further includes storing the HTPP at about −80° C. or lower until use. In some embodiments, the aliquot contains an amount of protein corresponding to a cell equivalent of at least about one million cells (e.g., about one million HUCPVCs). In some embodiments, the volume of the aliquot is about one milliliter (mL) or less.

In some embodiments, the HTPP contains an amount of protein corresponding to a cell equivalent of about one million cells per mL.

In some embodiments, the amount of protein in the HTPP is about 20 μM to about 80 μM per mL. In some embodiments, the amount of protein in the HTPP is about 45 μM per mL.

In another aspect, featured is a composition containing an HTPP produced according to the methods of the invention. In some embodiments, the composition further includes a pharmaceutically acceptable excipient. In some embodiments, the HTPP is diluted to form the composition.

In another aspect, featured is a method of treating a subject having an autoimmune or inflammatory disease or condition by administering to the subject a therapeutically effective amount of a composition of the invention (e.g., a composition containing an HTPP produced according to the methods of the invention). In some embodiments, the autoimmune or inflammatory disease or condition is selected from the group including achalasia, acne vulgaris, acute disseminated encephalomyelitis (ADEM), acute necrotizing hemorrhagic leukoencephalitis, Addison's disease, adjuvant-induced arthritis, adult Still's disease, agammaglobulinemia, alopecia areata, amyloidosis, ankylosing spondylitis, anti-GBM/anti-TBM nephritis, antiphospholipid syndrome (APS), atopic dermatitis, autoimmune angioedema, autoimmune aplastic anemia, autoimmune dysautonomia, autoimmune encephalomyelitis, autoimmune gastric atrophy, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune orchitis, autoimmune pancreatitis, autoimmune retinopathy, autoimmune thrombocytopenic purpura (ATP), autoimmune thyroid disease, autoimmune urticaria, axonal & neuronal neuropathy (AMAN), Balo disease, Behcet's disease, benign mucosal pemphigoid, bullous pemphigoid, Castleman disease, celiac disease, Chagas disease, chronic inflammatory demyelinating polyneuropathy (CIDP), chronic recurrent multifocal osteomyelitis (CRMO), Churg-Strauss syndrome, cicatricial pemphigoid, Crohn's disease, Cogan's syndrome, collagen-induced arthritis, cold agglutinin disease, congenital heart block, coxsackie myocarditis, CREST syndrome, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, epidermolysis bullosa, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalopathy, Hashimoto's thyroiditis, hemolytic anemia, Henoch-Schonlein purpura (HSP), herpes gestationis, Hidradenitis Suppurativa (Acne Inversa), hypogammaglobulinemia, idiopathic thrombocytopenic purpura (ITP), IgA nephropathy, IgG4-related sclerosing disease, inclusion body myositis, interstitial cystitis, inflammatory bowel disease, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease (LAD), lupus (Systemic Lupus Erythematosus), Lyme disease chronic, Meniere's disease, microscopic polyangiitis, mixed connective tissue disease (MCTD), Mooren's ulcer, Mucha-Habermann disease, multifocal motor neuropathy (MMN), multiple sclerosis, myasthenia gravis, myositis, narcolepsy, neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, pars planitis (peripheral uveitis), pemphigus, peripheral neuropathy, perivenous encephalomyelitis, pernicious anemia, POEMS syndrome, polyarteritis nodosa, polyglandular syndromes type I, II, III, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, pyoderma gangrenosum, pure red cell aplasia, Raynaud's phenomenon, reactive arthritis, reflex sympathetic dystrophy, relapsing polychondritis, restless legs syndrome, retroperitoneal fibrosis, rheumatic fever, rheumatoid arthritis, rosacea, sarcoidosis, Schmidt syndrome, scleritis, scleroderma, primary sclerosing cholangitis, chronic sclerosing sialadenitis, Sjogren's syndrome, sperm & testicular autoimmunity, stiff person syndrome, subacute bacterial endocarditis (SBE), Susac's syndrome, Sweet syndrome, sympathetic ophthalmia, Takayasu's arteritis, thrombocytopenic purpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, vitiligo, and Vogt-Koyanagi-Harada Disease. In some embodiments, the autoimmune or inflammatory disease or condition is scleroderma. In some embodiments, the autoimmune or inflammatory disease or condition is rheumatoid arthritis. In some embodiments, the autoimmune or inflammatory disease or condition is Sjogren's syndrome.

In another aspect, featured is a method of treating a subject having an autoimmune or inflammation-related skin disease or condition by administering to the subject a therapeutically effective amount of a composition of the invention (e.g., a composition containing an HTPP produced according to the methods of the invention). In some embodiments, the autoimmune or inflammation-related skin disease condition is atopic dermatitis, psoriasis, scleroderma, dermatomyositis, epidermolysis bullosa, pemphigus, bullous pemphigoid, alopecia areata, ocular cicatricial pemphigoid, dermatitis herpetiformis, linear IgA disease, dermatomyositis, lupus, vasculitis, Bechet's disease, lichen planus, rosacea, acne vulgaris, pyoderma gangrenosum, Hidradenitis Suppurativa, Sweet syndrome, autoimmune urticaria, a rash, or itchy skin.

In another aspect, featured is a method of improving cosmetic appearance in a subject in need thereof by administering to the subject a composition of the invention (e.g., a composition containing an HTPP produced according to the methods of the invention). In some embodiments, the method includes comprises reducing skin redness, reducing skin dullness, reducing skin dryness, reducing the appearance of fine lines and wrinkles, reducing the appearance of dark spots, reducing hyperpigmentation, reducing uneven skin texture, reducing acne, reducing the appearance of a scar or pockmark, reducing the appearance of a stretch mark, reducing pore size, firming the skin, brightening the skin, hydrating the skin, reducing skin irritation, soothing sunburned skin, increasing the production of collagen, and/or reducing collagen degradation.

In another aspect, featured is a method of reducing local inflammation in a subject in need thereof by administering to the subject an effective amount of a composition of the invention (e.g., a composition containing an HTPP produced according to the methods of the invention). In some embodiments, the subject has inflammation of a bursa, inflammation of a tendon, inflammation of a joint, inflammation of the jaw, inflammation of a gland, back pain, neck pain, sciatica, trigger finger, carpal tunnel syndrome or another entrapment syndrome, synovitis, rotator cuff syndrome, impingement syndrome, alopecia, frozen shoulder syndrome, fasciitis, or gout. In some embodiments, inflammation of the joint is associated with arthritis.

In another aspect, featured is a method of increasing or inducing angiogenesis (e.g., vascularization) in a subject in need thereof by administering to the subject an effective amount of a composition of the invention (e.g., a composition containing an HTPP produced according to the methods of the invention). In some embodiments, the subject has a cardiovascular disease, a wound, abnormal vasculature, poor vascularization, has received a tissue transplant, or is at risk of losing a limb.

In some embodiments of any of the foregoing aspects, the composition is administered by injection. In some embodiments of any of the foregoing aspects, the composition is administered topically. In some embodiments of any of the foregoing aspects, the composition is administered by inhalation (e.g., the composition is formulated as an aerosol or formulated for nebulization). In some embodiments of any of the foregoing aspects, the composition is administered locally to a site of inflammation.

In some embodiments of any of the foregoing aspects, the composition is diluted prior to administration to a subject.

In another aspect, featured is a method of improving the health, proliferation, and/or longevity of cultured HUCPVCs by contacting the cultured HUCPVCs with an effective amount an HTPP produced according to the methods of the invention or a composition of the invention. In some embodiments, the method improves HUCPVC proliferation.

In another aspect, featured is a cosmetic composition comprising an HTPP produced according to the methods of the invention. In some embodiments, the cosmetic composition is formulated as a face or body cream, face or body lotion, ointment, oil, serum, essence, gel, mist (e.g., spray), mask, foundation, blush, eyeshadow, mascara, eyeliner, lip product, setting powder, setting spray, tinted moisturizer, BB cream, CC cream, primer, tinted under-eye cream, concealer, nail product, light-protective product, after sun product, skin cleansing product, bath product, skin-tanning product, deodorant, antiperspirant, hair removal product, shaving product, fragrance, insect repellant, or hair care product. In some embodiments, the cosmetic composition further comprises a cosmetic active ingredient. In some embodiments, the cosmetic active ingredient is tocopherol, tocopherol acetate, tocopherol palmitate, deoxyribonucleic acid, retinol, bisabolol, allantoin, phytantriol, panthenol, an amino acid, an essential oil, a plant extract, a vitamin complex, a retinoid, Vitamin C, Vitamin A, an alpha-hydroxy acid, a beta-hydroxy acid, a glycolic acid, a kojic acid, an ascorbic acid, a hyaluronic acid, alpha-lipolic acid, hydroquinone, copper peptide, Vitamin E, dimethylaminoethanol (DMAE), niacinamide, a ceramide, a pseudoceramide, a curcuminoid, or an antioxidant. In some embodiments, the cosmetic composition further includes a fatty alcohol, fatty acid ester, natural or synthetic triglyceride, pearlescent wax, hydrocarbon oil, silicone or siloxane, fluorinated or perfluorinated oil, emulsifier, superfatting agent, surfactant, consistency regulator/thickener, theology modifier, polymer, deodorizing active ingredient, anti-dandruff agent, film former, hydrotropic agent, preservative, bacteria inhibiting agent, perfume oil, colorant, polymeric bead, hollow sphere, solubilizer, structurant, opacifier, complexing agent, or insect repellant. In some embodiments, the cosmetic composition is formulated for topical administration.

In another aspect, featured is a dermatologic composition comprising an HTPP produced according to the methods of the invention. In some embodiments, the cosmetic composition is formulated as a face or body cream, lotion, ointment, gel, or spray. In some embodiments, the cosmetic composition further includes a dermatologic active ingredient. In some embodiments, the dermatologic active ingredient is a steroid, a corticosteroid, coal tar, a retinoid, a calcineurin inhibitor, a vitamin D analog, doxepin, benzoyl peroxide, azeliac acid, dapsone, a phosphodiesterase-4 inhibitor, anthralin, an anti-bacterial agent, or an anti-fungal agent. In some embodiments, the dermatologic composition is formulated for topical administration.

In another aspect, featured is a method of making a perivascular tissue lysate, the method including the steps of (a) obtaining one or more umbilical cord vessels; (b) removing Wharton's Jelly adjacent to the umbilical cord vessels; (c) homogenizing the Wharton's Jelly to produce a homogenate; (d) centrifuging the homogenate to produce a supernatant and a pellet; (e) collecting the supernatant in a collection tube; (0 repeating steps (d) and (e) using the same collection tube; (g) centrifuging the collection tube to produce a final supernatant; and (h) sterilizing the final supernatant to produce the perivascular tissue lysate. In some embodiments, the one or more umbilical cord vessels are rinsed to remove blood prior to step (b). In some embodiments, the one or more umbilical cord vessels are rinsed with phosphate buffered saline. In some embodiments, blood-contaminated Wharton's Jelly is discarded prior to step (c). In some embodiments, the Wharton's Jelly is weighed prior to step (c) and the method further includes adding saline or basal medium to the Wharton's Jelly to a final tissue weight (g):volume of saline (mL) ratio of 1:3. In some embodiments, the final supernatant is sterilized using a 0.22 μM filter. In some embodiments, the one or more umbilical cord vessels is freshly isolated from an umbilical cord. In some embodiments, the one or more umbilical cord vessels is frozen. In some embodiments, the method further includes a step of thawing the frozen umbilical cord vessels prior to step (b). In some embodiments, the frozen umbilical cord vessels are thawed in a water bath at about 37° C. In some embodiments, the centrifuging of step (d) and step (g) is performed at 500×g for about 15 minutes at about 4° C. In some embodiments, the method further includes aliquoting the perivascular tissue lysate after step (h). In some embodiments, the method further includes storing the perivascular tissue lysate at −80° C. or lower until use. In some embodiments, the one or more umbilical cord vessels comprise two arteries and one vein.

In another aspect, featured is a method of making a mesenchymal stem cell (MSC) lysate, the method including the steps of (a) obtaining cultured MSCs grown on a surface of a vessel; (b) aspirating a cell culture medium from the vessel; (c) contacting the cultured MSCs with trypsin until the MSCs detach from the surface of the vessel; (d) flushing the MSCs from the surface of the vessel using a complete medium; (e) collecting the complete medium containing the MSCs in a tube; (f) centrifuging the tube containing the MSCs; (g) aspirating the supernatant, and (i) performing three freeze-thaw cycles prior to resuspending the MSCs to one million cells per mL; or (ii) resuspending the MSCs in saline solution to one million cells per mL prior to performing three rounds of homogenization on ice; (h) centrifuging the solution containing the MSCs; and (i) collecting and sterilizing the supernatant to produce the MSC lysate.

In another aspect, featured is a method of making a mesenchymal stem cell (MSC) lysate, the method including the steps of (a) obtaining cultured MSCs grown on a surface of a vessel (b) aspirating a cell culture medium from the vessel; (c) adding saline to the vessel and scraping the MSCs off of the surface using a cell scraper; (d) collecting the saline containing the MSCs in a tube; (e) centrifuging the tube containing the MSCs; (0 aspirating the supernatant, and (i) performing three freeze-thaw cycles prior to resuspending the MSCs to one million cells per mL; or (ii) resuspending the MSCs in saline solution to one million cells per mL prior to performing three rounds of homogenization on ice; (g) centrifuging the solution containing the MSCs; and (h) collecting and sterilizing the supernatant to produce the MSC lysate.

In another aspect, featured is a method of making a mesenchymal stem cell (MSC) lysate, the method including the steps of (a) obtaining cryopreserved MSCs; (b) thawing the MSCs; (c) adding basal medium to the MSCs to achieve a total volume of 4 mL in a tube; (d) centrifuging the tube containing the MSCs; (e) aspirating the supernatant, and (i) performing three freeze-thaw cycles prior to resuspending the MSCs to one million cells per mL; or (ii) resuspending the MSCs in saline solution to one million cells per mL prior to performing three rounds of homogenization on ice; (g) centrifuging the solution containing the MSCs; and (h) collecting and sterilizing the supernatant to produce the MSC lysate.

In some embodiments of any of the foregoing aspects, the supernatant is sterilized using a 0.22 μM filter. In some embodiments of any of the foregoing aspects, a cell count is performed prior to centrifuging the tube containing the MSCs. In some embodiments of any of the foregoing aspects, the centrifuging of the tube or the solution containing the MSCs is performed at 290×g for about 10 minutes at about 4° C. In some embodiments of any of the foregoing aspects, the freeze-thaw cycles of step (i) are performed by freezing the cells at −20° C. or lower and thawing the cells at about 25° C. to about 37° C. In some embodiments of any of the foregoing aspects, the method further includes aliquoting the MSC lysate after sterilization. In some embodiments of any of the foregoing aspects, the method further includes storing the MSC lysate at −80° C. or lower until use.

In some embodiments of any of the foregoing aspects, the MSC is a human umbilical cord perivascular cell (HUCPVC).

In some embodiments of any of the foregoing aspects, the MSC is an MSC isolated from bone marrow, adipose tissue, amniotic fluid, amniotic membrane, dental tissue, endometrium, limb bud, menstrual blood, peripheral blood, umbilical cord blood, placenta, fetal membrane, embryonic yolk sac, salivary gland, skin, foreskin, synovial fluid, or sub-amniotic umbilical cord lining membrane.

In another aspect, featured is a lysate made by any one of the methods of the invention.

In another aspect, featured is a kit including an HTPP produced according to the methods of the invention, a composition of the invention (e.g., a composition including an HTPP produced according to the methods of the invention), a cosmetic composition of the invention, a dermatologic composition of the invention, or materials for producing an HTPP, composition, cosmetic composition, or dermatologic composition of the invention. In some embodiments, the materials include isolated umbilical cord vessels, isolated perivascular tissue, or isolated HUCPVCs and instructions for producing an HTPP of the invention. In some embodiments, the kit includes a composition of the invention (e.g., a composition including an HTPP produced according to the methods of the invention), a cosmetic composition of the invention, or a dermatologic composition of the invention and instructions for use thereof.

In another aspect, featured is a method of producing an HTPP, the method including the steps of a) obtaining HUCPVCs; b) homogenizing the HUCPVCs, optionally in saline, wherein the homogenate comprises exosomes and/or vesicles from the HUCPVCs; and c) filtering said homogenate to produce the HTPP. In some embodiments, the HTPP comprises about 200,000 to about 5000 million HUCPVC equivalents. In some embodiments, the HTPP comprises a volume of from about 0.01 mL to about 1 L.

In another aspect, featured is a composition produced by the foregoing methods. In some embodiments, the composition contains from about 200,000 to about 1 million HUCPVC equivalents in a volume of about 1 mL or less.

In another aspect, featured is a method of treating a disease in a subject by administering a composition of the invention to the subject.

In some embodiments of any of the foregoing aspects, the subject is a human.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, “administration” refers to providing or giving a subject a therapeutic agent (e.g., a perivascular stem cell or tissue lysate described herein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by local injection while a second therapeutic agent of the combination may be administered orally.

As used herein, the terms “effective amount,” “therapeutically effective amount,” and a “sufficient amount” of a composition described herein refer to a quantity sufficient to, when administered to the subject in need thereof, including a mammal, for example a human, effect beneficial or desired results, including clinical results, and, as such, an “effective amount” or synonym thereto depends upon the context in which it is being applied. For example, in the context of treating inflammation, it is an amount of the composition sufficient to achieve a treatment response as compared to the response obtained without administration of the composition. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition of the present disclosure is an amount which results in a beneficial or desired result in a subject as compared to a control. Note that when a combination of active ingredients is administered, the effective amount of the combination may or may not include amounts of each ingredient that would have been effective if administered individually. As defined herein, a therapeutically effective amount of a composition of the present disclosure may be readily determined by one of ordinary skill by routine methods known in the art.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., a marker of inflammation) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are topical, epicutaneous, inhalational, intra-articular, intrathecal, intravaginal, intravitreal, intrauterine, intra-lesional administration, lymph node administration, intratumoral administration, intramuscular administration, epidural administration (e.g., administration to the spinal cord), and administration to a mucous membrane of the subject, wherein the administration is intended to have a local and not a systemic effect.

The terms “perivascular stem cell lysate” and “HUCPVC” lysate, used interchangeably herein, refer to a lysate prepared from stem cells obtained from the perivascular region of human umbilical cord using the methods described herein.

As used herein, the terms “mesenchymal stem cell lysate” and “MSC lysate” refer to a lysate prepared from MSCs obtained from a source other than the perivascular region of umbilical cord using the same methods described for the preparation of a perivascular stem cell lysate described herein.

As used herein, the terms “perivascular tissue lysate” and “PTL” refer to a lysate prepared using tissue and/or cells from the umbilical cord perivascular region of Wharton's Jelly, e.g., using the methods described herein.

As used herein, the terms “HUCPVC total protein product” and “HTPP” refer to a product prepared from human umbilical cord perivascular cells (HUCPVCs) using the methods described herein. This product includes proteins secreted by HUCPVCs (e.g., secreted in exosomes or vesicles) in addition to HUCPVC intracellular and transmembrane proteins.

As used herein, the term “MSC total protein product” refers to a product prepared from MSCs using the methods described for the preparation of an HTPP described herein. This product includes proteins secreted by MSCs (e.g., secreted in exosomes or vesicles) in addition to MSC intracellular and transmembrane proteins.

As used herein, the term “pharmaceutical composition” refers to a mixture containing a therapeutic agent (e.g., an HTPP as described herein), optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and/or other problem complications commensurate with a reasonable benefit/risk ratio.

As used herein, the term “proliferation” refers to an increase in cell numbers through growth and division of cells.

As used herein, the terms “subject” and “patient” refer to an animal (e.g., a mammal, such as a human). A subject to be treated according to the methods described herein may be one who has a disease or condition associated with inflammation, or a skin condition, or one at risk of developing the disease or condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

### BRIEF DESCRIPTION OF THE DRAWINGS

FIG. 1 is a graph showing the effect of perivascular tissue lysate (PTL) on mouse hindpaw circumference in a model of inflammation. As shown in in FIG. 1, a significant reduction in hindpaw circumference was observed 48 hours after mice were treated with PTL (gray) and inflammatory agent carrageenan compared to mice treated with saline (control, black) and carrageenan. A decrease in paw circumference was also observed at the 24 hour time point.

FIG. 2 is a graph showing the effect of PTL on the expression of myeloperoxidase (MPO) in a model of inflammation. As shown in in FIG. 2, a marked decrease in MPO level was observed in in the injected paw of mice treated with PTL (gray) and inflammatory agent carrageenan compared to the MPO level in the injected paw of mice treated with saline (control, black) and carrageenan. A decrease in MPO is indicative of a reduction in neutrophil infiltration.

FIG. 3 is a graph showing the effect of PTL and human umbilical cord perivascular cell (HUCPVC) total protein product (HTPP) on pro-inflammatory cytokine tumor necrosis factor-alpha (TNFα) concentration in a model of inflammation. As shown in in FIG. 3, TNFα concentration was reduced in the injected paw of mice treated with PTL and inflammatory agent carrageenan compared to the TNFα concentration in the injected paw of mice treated with saline and carrageenan at four 4 hours (black) after injection. The TNFα concentration was further reduced in the injected paw of mice treated with PTL and carrageenan 24 hours (gray) after injection. The concentration of TNFα in the injected paw of mice treated with HTPP and carrageenan was lower at 24 hours after injection than the concentration of TNFα observed in the injected paw of mice treated with PTL and carrageenan.

FIG. 4 is a graph quantifying the effect of perivascular tissue lysate on HUCPVC health and longevity after four days in culture. Cell proliferation in HUCPVC cultures cultured with perivascular tissue lysate at concentrations of 1% and 0.1% was found to be more than three times greater than control.

FIG. 5 is a graph quantifying the effect of HTPP on HUCPVC health and longevity after four days in culture. Cell proliferation in HUCPVC cultures treated with HTPP at a concentration of 1% was found to be more than three times greater than control, and cell proliferation in HUCPVC cultures treated with HTPP at concentrations of 0.1% and 0.01% was found to be more than two and a half times greater than control.

FIG. 6 is a graph quantifying the effect of HTPP on HUCPVC health and longevity after seven days in culture. After seven days in culture, HUCPVC proliferation in cultures grown with HTPP at concentrations of 0.1% and 0.01% was found to be more than fourteen times greater than control (basal culture medium).

FIG. 7A is a chart showing the percentage of proteins of each type identified in whole umbilical cord tissue lysate. Whole umbilical cord lysate contains fewer proteins of interest compared to perivascular tissue lysate and contains a smaller proportion of immune response proteins.

Perivascular tissue lysate contains a higher proportion of anti-inflammatory proteins, proteins related to immune response, and proteins related to angiogenesis compared to whole cord lysate.

FIG. 7B is a chart showing the percentage of proteins of each type identified in perivascular tissue lysate. Perivascular tissue lysate contains a greater number of anti-inflammatory proteins, proteins related to immune response, and proteins related to angiogenesis compared to whole cord lysate, as well as a greater proportion of immune response proteins.

FIG. 8 is a graph showing the effect of HTPP on the number of aspirated cells from the synovial joint space in a model of temporomandibular jaw inflammation. As shown in in FIG. 6, significantly fewer cells were aspirated from the synovial space 4 hours after rats were treated with HTPP (gray) and inflammatory agent carrageenan compared to rats treated with saline (control, black) and carrageenan (p=0.01).

### DETAILED DESCRIPTION

Described herein are compositions containing umbilical cord perivascular cell proteins, such as umbilical cord perivascular cell total protein products (e.g., compositions that contain secreted, transmembrane, and intracellular proteins produced by cultured perivascular cells) or perivascular tissue lysates (e.g., lysates derived from intact perivascular Wharton's Jelly), or compositions containing proteins produced by cultured mesenchymal stem cells (MSCs), such as cultured MSC lysates or total protein products (e.g., lysates or total protein products derived from cultured umbilical cord perivascular stromal cells or other types of MSCs). These lysates and total protein products also contain other cellular components, such as cell membrane fragments and intracellular cellular structures. These compositions can be incorporated into pharmaceutical compositions, cosmetic compositions, and dermatological products to treat autoimmune or inflammatory diseases or conditions, inflammation-related skin diseases and conditions, diseases or conditions characterized by local inflammation, skincare concerns, dermatological indications, and diseases or conditions characterized by a lack of angiogenesis or a need for the formation of additional blood vessels. The compositions can also be applied to cultured cells, such as cultured umbilical cord perivascular cells, to improve the health and longevity of the cultured cells. The invention also features methods of producing the lysates or total protein products containing umbilical cord perivascular cell or MSC proteins.

Human Umbilical Cord Perivascular Cells (HUCPVCs)

Human umbilical cord perivascular cells (HUCPVCs) are a non-hematopoietic, mesenchymal, population of multipotent cells obtained from the perivascular region within the Wharton's Jelly of human umbilical cords (see, e.g., Sarugaser et al., “Human umbilical cord perivascular (HUCPV) cells: A source of mesenchymal progenitors,” Stem Cells 23:220-229 (2005)). U.S. Patent Application Publication 2005/0148074, U.S. Pat. No. 8,278,102, and International Patent Application Publication WO 2007/128115 describe methods for the isolation and culture of HUCPVCs and are incorporated by reference herein. HUCPVCs are further characterized by relatively rapid proliferation, exhibiting a doubling time, in each of passages 2-7, of about 20 hours (serum dependent) when cultured under standard adherent conditions. Phenotypically, the HUCPVCs are characterized, at harvest, as Oct 4−, CD14−, CD19−, CD34−, CD44+, CD45−, CD49e+, CD90+, CD105(SH2)+, CD73(SH3)+, CD79b−, HLA-G−, CXCR4+, and c-kit+. In addition, HUCPVCs are positive for CK8, CK18, CK19, PD-L2, CD146 and 3G5 (a pericyte marker), at levels higher relative to cell populations extracted from Wharton's Jelly sources other than the perivascular region.

The present disclosure is based, at least in part, on the discovery that total protein-containing products from mesenchymal stem cells, such as HUCPVCs (e.g., products that include intracellular and membrane-bound cellular proteins, as well as secreted proteins present in exosomes or vesicles that are secreted by the cell), have beneficial therapeutic properties. In particular, the total protein-containing products can be prepared using tissue from the perivascular region of vessels in Wharton's Jelly from the umbilical cord (e.g., the perivascular region containing HUCPVCs) or from cultured HUCPVCs. These products were found to contain over 600 proteins or protein fragments and to include small particles between about 20 to about 200 nanometers (nm) in size. In addition, incubation of HUCPVCs with saline was found to allow for the production of a more complete protein product containing secreted, intracellular, and transmembrane perivascular cell proteins. Unlike other connective tissues, Wharton's Jelly contains no blood vessels, nerves, lymph vessels, or other tissues. Therefore, perivascular tissue and stem cell-based products containing HUCPVC proteins and microparticles can be prepared from Wharton's jelly reasonably free from contamination from other cell or tissue types. These products also contain extracellular matrix (ECM) proteins produced by the HUCPVCs, which may contribute to or enhance their therapeutic effects. The perivascular tissue and stem cell products were found to exhibit anti-inflammatory properties in vivo, and to promote growth and longevity of cultured cells in vitro. These products were also enriched for therapeutic proteins compared to products (e.g., lysates) prepared from whole umbilical cord. While previous therapies have employed isolated HUCPVCs or supernatant derived from cultured HUCPVCs, the products of the present application contain both HUCPVC proteins and ECM proteins, and, in the case of total protein products, intracellular, transmembrane, and secreted HUCPVC proteins, which provide improved therapeutic benefits compared to cells or supernatant alone. Methods for preparing these products and uses for such products are described herein.

Compositions Containing Perivascular Tissue Lysates, MSC Total Protein Products, and/or MSC Lysates

Featured are total protein products that include intracellular, transmembrane, and secreted proteins from MSCs, such as HUCPVCs. These protein products, described as “total protein products” due to their inclusion of intracellular, transmembrane, and secreted proteins produced by MSCs (e.g., HUCPVCs), have a distinct advantage compared to standard stem cell-based compositions. Typical MSC-based compositions, including MSC conditioned medium, intact MSCs, and MSC lysates, fail to adequately capture at least one class of proteins. For example, MSC conditioned medium excludes transmembrane and intracellular MSC proteins, MSC lysates exclude secreted proteins, and intact MSCs communicate with their external environment using secreted and transmembrane proteins, failing to expose neighboring cells to intracellular proteins. The total protein products described herein are produced using methods that allow for the recovery of all three types of proteins due to the homogenization of the cells together with the materials (e.g., exosomes and vesicles) secreted by the cells, and accordingly, the total protein products include more protein, and may include a wider variety of proteins, than typical MSC-based compositions. Accordingly, the total protein products can be used to treat a variety of indications and have been found to exhibit beneficial results in both in vivo and in vitro applications.

Also featured are compositions containing perivascular tissue lysates (e.g., lysates derived from intact perivascular Wharton's Jelly) and/or MSC lysates (e.g., lysates derived from cultured HUCPVCs or other types of cultured MSCs). These lysates contain MSC proteins (e.g., HUCPVC proteins) and ECM proteins and exhibit therapeutic properties in vivo. We have discovered that perivascular tissue lysates contain nearly twice as many therapeutic proteins of interest as whole umbilical cord lysates and that compositions containing perivascular tissue lysates can both reduce inflammation and promote perivascular cell proliferation. Based on these findings, perivascular tissue lysates may also provide a number of therapeutic benefits, as is discussed below.

Production of Perivascular Total Protein Products

Perivascular total protein products (e.g., HUCPVC total protein products, or HTPPs) can be prepared using cultured perivascular stromal cells. Perivascular stromal cells can be isolated from the Wharton's Jelly attached to either fresh or frozen umbilical cord vessels (e.g., human umbilical cord vessels) as described herein below and expanded in culture. Methods of isolating HUCPVCs from fresh or frozen umbilical cord vessels are also described in, e.g., U.S. Pat. Nos. 7,547,546 and 8,278,102, incorporated herein by reference. Once in culture, the perivascular stromal cells can be passaged one or more times (e.g., 1, 2, 3, 4, 5, 6, 7, 8, 9, or 10 times).

The perivascular stromal cells can be harvested after one or more passages and used to produce a total protein product (e.g., the cells can be harvested at passage 1 (P1), P2, P3, P4, P5, P6, P7, P8, P9, or P10, such as at P3). Perivascular stromal cells can be harvested by aspirating the spent medium from the cultured cells, trypsinizing the cells (e.g., adding a trypsin-containing product to the cells, such as about 10 to about 15 mL of TrypLE, and incubating the cells for about 5 to about 10 minutes (e.g., 10 minutes) at about 35° C. to about 37° C.). Once the cells have completely detached from the culture vessel (e.g., a flask or plate), they can be flushed from the vessel using complete medium and a pipette (e.g., about 10 to about 15 mL of complete medium, such as 12 mL of complete medium) and transferred to a 50 mL tube. Next, the cells can be centrifuged to create a pellet (e.g., at about 290×g at about 4° C. for about 10 minutes) followed by resuspension and cell counting (e.g., using a hemocytometer or ViCell). Once cell counts have been determined, the perivascular stromal cells can be seeded for further expansion (e.g., at about 2000 cells/cm²), cryopreserved for future use, or used to produce a total protein product.

Whether cells are to be cryopreserved or immediately used to produce a total protein product, they are first centrifuged to create a pellet (e.g., at about 290×g at about 4° C. for about 10 minutes). The perivascular stromal cells can then be resuspended in an appropriate solution. If the perivascular stromal cells are to be used immediately to produce a total protein product, they are resuspended in saline (e.g., about 0.5 mL to about 1.5 mL saline, such as 1 mL saline) and counted (e.g., using a hemocytometer or ViCell and a 1:10 dilution). Alternatively, if cryopreserved perivascular cells used to produce the total protein product, they can first be thawed until a small sliver of ice remains (e.g., in an about 30° C. to about 37° C. water bath for about 2 minutes). Complete medium can then be added dropwise to the vial containing the cells (e.g., about 1 mL), and after a two-minute waiting period at room temperature, the contents of the vial can be transferred to a 15 mL tube and additional complete medium can be added to the 15 mL tube (e.g., about 2 mL). After another wait at room temperature (e.g., about five minutes), the perivascular stromal cells can be centrifuged to create a pellet (e.g., at about 290×g at about 4° C. for about 10 minutes), resuspended in saline (e.g., about 0.5 mL to about 1.5 mL saline, such as 1 mL saline), and counted (e.g., using a hemocytometer or ViCell and a 1:10 dilution).

The perivascular cells are then incubated in the saline (0.9% sodium chloride in sterile water) at about 37° C. for about 15 minutes to about 24 hours (e.g., 15 minutes, 30 minutes, 45 minutes, or 1, 2, 3, 4, 5, 6, 7, 8, 9, 10, 11, 12, 13, 14, 15, 16, 17, 18, 19, 20, 21, 22, 23, or 24 hours, e.g., about 3 to about 8 hours, about 4 to about 7 hours, or about 6 hours). Without wishing to be bound by theory, incubating the perivascular cells in saline for a period of hours allows the cells to release secreted proteins (e.g., in exosomes and/or vesicles). Following the incubation in saline, the cells remain in the saline with the secreted proteins (e.g., the exosomes and/or vesicles) and are homogenized (e.g., using four rounds of 30 seconds of homogenization, e.g., bead mill-based homogenization, with 1-minute intervals on ice). After homogenizing the cells, saline is added for a final concentration of approximately 1 million cells (corresponding to 1 million cell equivalents) per mL. The supernatant is then filter sterilized (e.g., using a syringe filter, such as an about 0.2 μm to an about 0.45 μm syringe filter, such as a 0.22 μm syringe filter, or a vacuum filter), and it is the sterilized material that constitutes the total protein product. The total protein product can be used immediately or aliquoted (e.g., in 5 μL, 10 μL, 20 μL, 25 μL, 50 μL, 100 μL, 250 μL, 500 μL, 750 μL, 1 mL, 1.5 mL, 2 mL, 2.5 mL, 5 mL, or 10 mL aliquots) and frozen (e.g., stored at a temperature of −80° C. or lower, e.g., at −80° C., −120° C., or −140° C.) for later use. The HTPP contains all three classes of proteins: intracellular, transmembrane, and secreted (e.g., proteins contained in the exosomes and/or vesicles) in addition to other cellular components, such as cell membrane fragments and intracellular structures.

In an alternative methodology, HTPP can be produced without culturing. In this method, HUCPVCs are collected directly from the perivascular tissue attached to the umbilical cord vessels and homogenized with exosomes and/or vesicles in a small volume (e.g., to produce a homogenized solution having at least 500,000 cell equivalents, such as about 500,000 cell equivalents to about 2 million cell equivalents in a volume of 2 mL or less, such as in a volume of 2 mL, 1.5 mL, 1 mL or 0.5 mL). The resulting homogenate is a HTPP containing all three classes of proteins: intracellular, transmembrane, and secreted (e.g., proteins contained in the exosomes and/or vesicles). The HTPP also contains other cellular components, such as cell membrane fragments and intracellular structures.

Production of Perivascular Tissue Lysate

Perivascular tissue lysate can be prepared using fresh or frozen human umbilical cord vessels (e.g., arteries and/or veins). If frozen umbilical cord vessels are used to produce perivascular tissue lysate, the lysate can be produced once the vessels are thawed. Perivascular tissue lysates can also be prepared using umbilical cord vessels from other placental mammals.

An exemplary method for producing perivascular tissue lysate from frozen human umbilical cord vessels includes a first step of thawing (e.g., in an about 30° C. to about 37° C. water bath for about 15 to 20 minutes) frozen human umbilical cord vessels (e.g., one 15 mL cryopod containing 2 arteries and 1 vein for each donor lot). Next, the vessels are transferred to a specimen container and rinsed with phosphate buffered saline (PBS, e.g., 100 to 250 mL PBS), which may be performed by adding PBS to the container, sealing the container (e.g., with a cap), and shaking the container to rinse out blood. The vessels can be rinsed an additional one or more times (e.g., 1, 2, 3, 4, or more additional rinses, e.g., once more for a total of two rinse steps) by transferring the vessels to a fresh specimen container and repeating the rinse step. Once the vessels have been rinsed, Wharton's jelly (WJ) can be removed by placing the vessels on a surface (e.g., a silicone mat) and stripping the WJ (e.g., using forceps). Any WJ that is contaminated by blood can be cut off and discarded, leaving strips of WJ that are free from blood contamination. The WJ strips can then be cut into small pieces (e.g., 0.5 cm to 2.0 cm in length), transferred to a specimen container, and weighed. Next, the WJ tissue can be transferred to a container for homogenization or blending (e.g., for manual homogenization, such as using a mortar and pestle, or for homogenization or blending using a machine, such as bead-based or blender-based homogenization). For example, the WJ tissue can be transferred to a blender cup (e.g., a Baby Bullet blender cup). The specimen container previously holding the WJ can then be rinsed with saline (e.g., about 5 mL to about 10 mL saline), which can then be collected and added to the container for homogenization or blending (e.g., the blender cup). Frozen saline can then be added to the container for homogenization or blending (e.g., the blender cup) to achieve a final tissue weight (g): volume of saline (mL) ratio of about 1:3. If the perivascular tissue lysate is to be used for an in vitro application (e.g., added to cultured cells), basal medium can be used instead of saline. The tissue can then be homogenized or blended (e.g., blended continuously for about 2 minutes in the Baby Bullet blender, or until the tissue is fully blended or homogenized), and the homogenized or blended material can be transferred to two fresh tubes for centrifugation in a refrigerated centrifuge (e.g., to two 15 or 50 mL tubes for centrifugation at about 500×g for about 15 to 20 minutes at about 4° C.). After centrifugation, the supernatant can be collected and pooled in a collection tube. The centrifugation and supernatant collection steps can then be repeated, with all of the collected supernatant added to the single collection tube. Next, the pellets can be spun at a higher speed in a refrigerated centrifuge (e.g., at about 3716×g for about 15 to 20 minutes at about 4° C.) and the resulting supernatant can be collected and added to the single collection tube. The pellets can be spun and the supernatant collected until there is no more supernatant separation. Once all of the supernatant has been collected, the single collection tube can be spun in the refrigerated centrifuge (e.g., at about 500×g for about 15 to 20 minutes at about 4° C.). After centrifugation, the supernatant from the collection tube can be collected and sterilized by filtration (e.g., through a 0.22 μM filter, such as a syringe filter or a vacuum filtration system). The resulting material is the perivascular tissue lysate, which can be used immediately or aliquoted (e.g., in 5 μL, 10 μL, 20 μL, 25 μL, 50 μL, 100 μL, 250 μL, 500 μL, 750 μL, 1 mL, 1.5 mL, 2 mL, 2.5 mL, 5 mL, or 10 mL aliquots) and frozen (e.g., stored at a temperature of −80° C. or lower, e.g., at −80° C., −120° C., or −140° C.) for later use.

Production of Perivascular Stem Cell Lysate

Lysate can also be prepared from cultured human umbilical cord perivascular stromal cells (HUCPVCs). HUCPVCs can be isolated from fresh or frozen human umbilical cord using established methods, such as those described in, e.g., U.S. Pat. Nos. 7,547,546 and 8,278,102, incorporated herein by reference, and subsequently cultured and expanded as described herein. Once the cells have passaged one or more times (e.g., 1, 2, 3, 4, 5, 6, 7, 8, 9, or 10 times), they can be harvested and used to generate a lysate (e.g., the cells can be harvested at passage 1 (P1), P2, P3, P4, P5, P6, P7, P8, P9, or P10, such as at P3).

Three different approaches that can be used to produce perivascular stem cell lysate are described herein.

In a first approach, expanded HUCPVCs can be harvested with trypsinization to produce a cell lysate. First, spent culture medium can be aspirated, then a pre-warmed trypsin-containing solution (e.g., TrypLE Express Enzyme) can be added to the HUCPVC culture e.g., (about 2 mL to about 10 mL per 75 cm², e.g., about 4 mL) and the cells can be incubated at about 37° C. for about 5 minutes to about 10 minutes. Next, the vessel (e.g., plate, dish, or flask) containing the HUCPVCs can be tapped gently to detach the cells and complete cell detachment can be confirmed visually using a microscope. Complete medium can then be added to the culture vessel and the HUCPVCs can be flushed off of the cell culture surface (e.g., a pipette can be used to flush the cells off the surface using the medium). The resulting cell suspension can be added to a collection tube and a cell count can be performed (e.g., using a hemocytometer and Trypan Blue) to determine the number of viable cells. The cells can then be spun in a refrigerated centrifuge at about 200×g to about 400×g, e.g., at 290×g for about 10 to about 15 minutes at 4° C. After centrifugation, the supernatant can be aspirated and the cells can be lysed, for example, using methods known in the art. Two methods for cell lysis are described:

Method 1: Perform 3 freeze-thaw cycles by freezing the cells at −20° C. or lower (e.g., −20° C., −25° C., −30° C., −35° C., −40° C., or lower) and thawing the cells at about 25° C. to about 37° C., then re-suspend in saline solution to about one million cells per mL.

Method 2: Re-suspend cells in saline solution to about one million cells per mL and homogenize (e.g., using a manual homogenizer or a bead homogenizer) for three 30 to 45 second rounds with a one to two-minute interval on ice.

In a second approach, expanded HUCPVCs can be harvested without trypsinization (e.g., using mechanical force to separate the cells from the vessel) to produce a cell lysate. First, spent culture medium can be aspirated, then the vessel (e.g., plate, dish, or flask) containing the HUCPVCs can be rinsed twice with PBS (about 5 mL to about 10 mL, e.g., about 8 mL per 75 cm²). Next, saline solution can be added to the vessel (about 2 mL to about 12 mL, e.g., about 4 mL per 75 cm²) and the cells can be detached from the vessel using mechanical force (e.g., using a cell scraper). Complete cell detachment can be confirmed visually using a microscope. The cell suspension can then be added to a collection tube and the cells can be lysed, for example, using methods known in the art. Two methods for cell lysis are described:

Method 1: Perform 3 freeze-thaw cycles by freezing the cells at −20° C. or lower (e.g., −20° C., −25° C., −30° C., −35° C., −40° C., or lower) and thawing the cells at about 25° C. to about 37° C., then re-suspend in saline solution to about one million cells per mL.

Method 2: Re-suspend cells in saline solution to about one million cells per mL and homogenize (e.g., using a manual homogenizer or a bead homogenizer) for three 30 to 45 second rounds with a one to two-minute interval on ice.

In a third approach, the cell lysate can be prepared from HUCPVCs that have been cryopreserved (e.g., as described in U.S. Pat. Nos. 7,547,546, 8,277,794, and 8,790,923). First, a vial of cells (e.g., a 1 mL vial) can be removed from liquid nitrogen and placed in a warm water bath (e.g., a water bath between about 33° C. and about 40° C., e.g., about 37° C.). The vial of cells can be thawed until a small sliver of ice remains (about 1-3 minutes, e.g., about 2 minutes) and then transferred to a biosafety cabinet. Next, about 1 mL of pre-warmed basal medium can be added dropwise to the vial of cells and incubated for about 1-5 min, e.g., about 2 min at room temperature (e.g., about 22° C. to about 27° C.). The content of the vial (now about 2 mL) can then be transferred to a 15 mL tube and enough basal medium can be quickly added to the tube to double the volume of medium in the tube (e.g., about 2 mL is added for a total of about 4 mL). The vial can be incubated at room temperature (e.g., about 22° C. to about 27° C.) for about 5 to 10 minutes, e.g., about 5 minutes, and then, optionally, a cell count can be performed (e.g., using a hemocytometer and Trypan Blue) to determine the number of viable cells. The cells can then be spun in a refrigerated centrifuge at about 200×g to about 400×g, e.g., at about 290×g, for about 10 to about 15 minutes at 4° C. After centrifugation, the supernatant can be aspirated and the cells can be lysed, for example, using methods known in the art. Two methods for cell lysis are described.

Method 1: Perform 3 freeze-thaw cycles by freezing the cells at −20° C. or lower (e.g., −20° C., −25° C., −30° C., −35° C., −40° C., or lower) and thawing the cells at about 25° C. to about 37° C., then re-suspend in saline solution to about one million cells per mL.

Method 2: Re-suspend cells in saline solution to about one million cells per mL and homogenize (e.g., using a manual homogenizer or a bead homogenizer) for three 30 to 45 second rounds with a one to two-minute interval on ice.

After the cells are lysed (e.g., using an approach such as those described above), the cell suspension can be spun at about 200×g to about 400×g, e.g., at about 290×g for about 10 to 20 minutes, e.g., about 15 minutes in a refrigerated centrifuge (e.g., at about 4° C.). After centrifugation, the supernatant can be collected and sterilized (e.g., through a 0.22 μM filter, such as a syringe filter or a vacuum filtration system). The resulting material is the perivascular stem cell lysate, which can be used immediately or aliquoted (e.g., in 5 μL, 10 μL, 20 μL, 25 μL, 50 μL, 100 μL, 250 μL, 500 μL, 750 μL, 1 mL, 1.5 mL, 2 mL, 2.5 mL, 5 mL, or 10 mL aliquots) and frozen (e.g., stored at a temperature of −80° C. or lower, e.g., at −80° C., −120° C., or −140° C.) for later use.

Production of Total Protein Products and Lysates from Other Types of Mesenchymal Stem Cells

In some embodiments, total protein products and/or lysates may be prepared from cultured MSCs other than HUCPVCs. Although other MSC-containing tissues (e.g., adipose tissue, bone marrow, dental tissue) may not be amenable to production of a tissue lysate due to the presence of non-MSCs in the tissue, isolated, cultured MSCs from these tissues can be used to prepare total protein products and/or cell lysates according to the methods described above. Tissues from which MSCs can be isolated and cultured for production of total protein products and/or cell lysates include bone marrow, adipose tissue, amniotic fluid, amniotic membrane, dental tissue, endometrium, limb bud, menstrual blood, peripheral blood, umbilical cord blood, placenta and fetal membrane, embryonic yolk sac, salivary gland, skin and foreskin, synovial fluid, and sub-amniotic umbilical cord lining membrane.

Uses of Perivascular Tissue Lysates, MSC Total Protein Products, and/or MSC Lysates

The MSC total protein products (e.g., HTPPs), perivascular tissue lysates, and/or MSC lysates (e.g., HUCPVC lysates) described herein can be used in a variety of applications. Perivascular tissue lysates were found to contain a large number of immune-related proteins (e.g., anti-inflammatory proteins and proteins that modulate the immune response) and were also found to reduce inflammation in vivo. Accordingly, MSC total protein products (e.g., HTPPs), perivascular stem cell lysates, and/or MSC lysates (e.g., HUCPVC lysates) or compositions including the lysates or total protein products can be used to treat autoimmune or inflammatory diseases or conditions and/or local inflammation. In addition, many of the proteins identified in the perivascular tissue lysates would be useful in skincare applications (e.g., anti-inflammatory proteins, angiogenic proteins, proteins that modulate the immune response, antioxidant proteins, and anti-aging proteins), indicating that MSC total protein products (e.g., HTPPs), perivascular stem cell lysates, and/or MSC lysates (e.g., HUCPVC lysates) may also be used for cosmetic and dermatological treatments. In addition, the lysates and total protein products have been found to promote beneficial effects on cell growth and longevity in culture, demonstrating their utility as cell culture reagents.

Use of Perivascular Tissue Lysates, MSC Total Protein Products, and/or MSC Lysates to Treat or Reduce Inflammation

Treatment of Autoimmune or Inflammatory Diseases or Conditions

The perivascular tissue lysates, MSC total protein products (e.g., HTPPs), and/or MSC (e.g., HUCPVC) lysates described herein can be used to treat an autoimmune or inflammatory disease or condition in a subject in need thereof by administering an effective amount of the lysate or total protein product, or a pharmaceutical composition containing the lysate or total protein product, to the subject. The method can include administering the perivascular tissue lysate, MSC total protein product (e.g., HTPP), and/or MSC (e.g., HUCPVC) lysate, or a pharmaceutical composition containing a lysate or total protein product described herein, locally (e.g., topically, by local injection, or by inhalation, e.g., inhalation into the lungs via nebulization or an aerosol) or systemically to the subject in a dose (e.g., effective amount) and for a time sufficient to treat the autoimmune or inflammatory disease or condition.

The methods described herein can be used to reduce or inhibit an immune response in a subject in need thereof, e.g., the subject has an autoimmune condition and is in need of inhibiting or reducing inflammation or an immune response against self- or auto-antibodies. The methods described herein can also include a step of selecting a subject in need of inhibiting an immune response, e.g., selecting a subject who has or who has been identified to have an autoimmune or inflammatory disease or condition.

The autoimmune or inflammatory disease or condition to be treated with a perivascular tissue lysate, MSC total protein product (e.g., HTPP), and/or MSC (e.g., HUCPVC) lysate, or a pharmaceutical composition containing a lysate or total protein product described herein, may be selected from achalasia, acne vulgaris, acute disseminated encephalomyelitis (ADEM), acute necrotizing hemorrhagic leukoencephalitis, Addison's disease, adjuvant-induced arthritis, adult Still's disease, agammaglobulinemia, alopecia areata, amyloidosis, ankylosing spondylitis, anti-GBM/anti-TBM nephritis, antiphospholipid syndrome (APS), atopic dermatitis, autoimmune angioedema, autoimmune aplastic anemia, autoimmune dysautonomia, autoimmune encephalomyelitis, autoimmune gastric atrophy, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune orchitis, autoimmune pancreatitis, autoimmune retinopathy, autoimmune thrombocytopenic purpura (ATP), autoimmune thyroid disease, autoimmune urticaria, axonal & neuronal neuropathy (AMAN), Balo disease, Behcet's disease, benign mucosal pemphigoid, bullous pemphigoid, Castleman disease, celiac disease, Chagas disease, chronic inflammatory demyelinating polyneuropathy (CIDP), chronic recurrent multifocal osteomyelitis (CRMO), Churg-Strauss syndrome, cicatricial pemphigoid, Crohn's disease, Cogan's syndrome, collagen-induced arthritis, cold agglutinin disease, congenital heart block, coxsackie myocarditis, CREST syndrome, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, epidermolysis bullosa, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalopathy, Hashimoto's thyroiditis, hemolytic anemia, Henoch-Schonlein purpura (HSP), herpes gestationis, Hidradenitis Suppurativa (Acne Inversa), hypogammaglobulinemia, idiopathic thrombocytopenic purpura (ITP), IgA nephropathy, IgG4-related sclerosing disease, inclusion body myositis, interstitial cystitis, inflammatory bowel disease, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease (LAD), lupus (e.g., Systemic Lupus Erythematosus), Lyme disease chronic, Meniere's disease, microscopic polyangiitis, mixed connective tissue disease (MCTD), Mooren's ulcer, Mucha-Habermann disease, multifocal motor neuropathy (MMN), multiple sclerosis, myasthenia gravis, myositis, narcolepsy, neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, pars planitis (peripheral uveitis), pemphigus, peripheral neuropathy, perivenous encephalomyelitis, pernicious anemia, POEMS syndrome, polyarteritis nodosa, polyglandular syndromes type I, II, III, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, pyoderma gangrenosum, pure red cell aplasia, Raynaud's phenomenon, reactive arthritis, reflex sympathetic dystrophy, relapsing polychondritis, restless legs syndrome, retroperitoneal fibrosis, rheumatic fever, rheumatoid arthritis, rosacea, sarcoidosis, Schmidt syndrome, scleritis, scleroderma, primary sclerosing cholangitis, chronic sclerosing sialadenitis, Sjogren's syndrome, sperm & testicular autoimmunity, stiff person syndrome, subacute bacterial endocarditis (SBE), Susac's syndrome, Sweet syndrome, sympathetic ophthalmia, Takayasu's arteritis, thrombocytopenic purpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, vitiligo, and Vogt-Koyanagi-Harada Disease. In some embodiments, the autoimmune or inflammatory disease or condition is scleroderma. In some embodiments, the autoimmune or inflammatory disease or condition is rheumatoid arthritis. In some embodiments, the autoimmune or inflammatory disease or condition is Sjogren's syndrome.

The autoimmune or inflammatory disease or condition may be recognized as an autoimmune or inflammation-related skin disease or condition. Exemplary autoimmune or inflammation-related skin diseases or conditions include atopic dermatitis, psoriasis, scleroderma, dermatomyositis, epidermolysis bullosa, pemphigus, bullous pemphigoid, alopecia areata, ocular cicatricial pemphigoid, dermatitis herpetiformis, linear IgA disease, dermatomyositis, lupus, vasculitis, Bechet's disease, lichen planus, rosacea, acne vulgaris, pyoderma gangrenosum, Hidradenitis Suppurativa, Sweet syndrome, autoimmune urticaria, a rash, and itchy skin.

Treatment of Local Inflammation

The perivascular tissue lysates, MSC total protein products (e.g., HTPPs), and/or MSC (e.g., HUCPVC) lysates described herein can also be formulated for injection to treat local inflammation. The lysates, total protein products, or pharmaceutical compositions containing the lysates or total protein products can be injected into or near a region of the body that is inflamed to reduce inflammation, pain, or swelling, or to prevent inflammation, swelling, or pain from worsening. Conditions for which local injection of a perivascular tissue lysate, MSC total protein product (e.g., HTPP), and/or an MSC (e.g., HUCPVC) lysate or a pharmaceutical composition containing such a lysate or total protein product can be used for treatment include, e.g., inflammation of a bursa (e.g., bursitis of the hip, knee, elbow, or shoulder), inflammation of a tendon (e.g., tendinitis, such as tennis elbow), inflammation of a joint (e.g., arthritis, such as osteoarthritis, rheumatoid arthritis, reactive arthritis, or psoriatic arthritis), inflammation of the jaw (e.g., as in temporomandibular joint (TMJ) disorders), inflammation of a gland (e.g., a salivary gland, such as in Sjogren's syndrome), back pain, neck pain, sciatica, trigger finger, carpal tunnel syndrome and other entrapment syndromes, synovitis, rotator cuff syndrome, impingement syndrome, alopecia, frozen shoulder syndrome, fasciitis, and gout. Perivascular tissue lysates, MSC total protein products (e.g., HTPPs), and/or MSC (e.g., HUCPVC) lysates or pharmaceutical compositions containing the lysates or total protein products may be given by intra-articular injection (e.g., into a joint), intramuscular injection (e.g., into a muscle), epidural injection (e.g., into the spine), or local injection to or near a region of inflammation. Perivascular tissue lysates, MSC total protein products (e.g., HTPPs), and/or MSC (e.g., HUCPVC) lysates or pharmaceutical compositions containing the lysates or total protein products may also be administered by inhalation (e.g., by nebulization or using an aerosol formulation) to treat lung inflammation.

The perivascular tissue lysates, MSC total protein products (e.g., HTPPs), and/or MSC (e.g., HUCPVC) lysates can also be used to increase or promote angiogenesis (e.g., vascularization). Accordingly, the lysates or total protein products described herein can be injected into regions of the body in need of additional blood flow or to treat subjects that may benefit from increased blood flow, such as subjects suffering from a cardiovascular disease, such as ischemia (e.g., to stimulate the growth of new vessels in ischemic tissue), subjects at risk of losing a limb, subjects who have received a tissue transplant (e.g., to promote vascularization in the tissue transplant), subjects having abnormal or poor vasculature, or subjects having a wound (e.g., for promoting wound healing).

The autoimmune or inflammatory diseases or conditions described above and the angiogenesis-related diseases or conditions described above can be treated with lysates produced from perivascular tissue and/or MSCs (e.g., HUCPVCs) or with MSC total protein products (e.g., HTPPs) prepared using MSCs. The lysates or protein products may be administered directly to a subject or formulated with a pharmaceutically acceptable carrier or excipient. The lysates or total protein products may also be formulated with other anti-inflammatory or immunosuppressive agents, or the lysates, total protein products, or lysate- or total protein product-containing pharmaceutical compositions may be administered in combination with other agents or medications used to treat the autoimmune, inflammatory, or angiogenesis-related disease or condition.

The lysates, total protein products, and compositions described herein may be administered to a subject in need thereof (e.g., a subject having a disease or condition described herein, e.g., an autoimmune or inflammatory disease or condition or an angiogenesis-related disease or condition) by a variety of routes, such as local administration to the site affected by the disease or condition (e.g., local administration to or near the site of inflammation or reduced angiogenesis, such as injection to a joint for treating arthritis (e.g., RA, psoriatic arthritis, osteoarthritis, or reactive arthritis), injection into the spine for treating sciatica, injection into an ischemic tissue for treating a subject having ischemia, or topical administration to a region affected by an inflammation-related skin disease or condition), intravenous, parenteral, intradermal, transdermal, intramuscular, intranasal, subcutaneous, percutaneous, intratracheal, intraperitoneal, intraarterial, intravascular, intrathecal, intra-articular, epidural, inhalation, perfusion, lavage, and oral administration. For topical administration, the lysates, total protein products, or compositions may be formulated as a cream, ointment, lotion, gel, suspension, emulsion, or spray. For inhalation, the lysates, total protein products, or compositions may be formulated as an aerosol or formulated for nebulization. The most suitable route for administration in any given case will depend on the particular disease or condition being treated, the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, weekly, daily, or twice a day). For local administration, the lysates, total protein products, and compositions may be administered topically, by injection, or by inhalation (e.g., to treat an autoimmune, inflammatory, or angiogenesis-related disease or condition).

Subjects that may be treated as described herein are subjects having a disease or condition described herein (e.g., an autoimmune or inflammatory disease or condition, an inflammation-related skin disease or condition, bursitis of the hip, knee, elbow, or shoulder; tendon inflammation, e.g., tendinitis, such as tennis elbow; joint inflammation, e.g., arthritis, such as osteoarthritis, rheumatoid arthritis, reactive arthritis, or psoriatic arthritis; jaw inflammation, e.g., TMJ disease; inflammation of a gland, e.g., a salivary gland, such as in Sjogren's syndrome; back pain; neck pain; sciatica; trigger finger; carpal tunnel syndrome and other entrapment syndromes; synovitis; rotator cuff syndrome; impingement syndrome; alopecia (e.g., alopecia areata); frozen shoulder syndrome; fasciitis; gout; or an angiogenesis-related disease or condition (e.g., a cardiovascular disease, such as ischemia, risk of loss of a limb, tissue transplant, abnormal or poor vasculature, or a wound (e.g., for promoting wound healing)). The lysates, total protein products, compositions, and methods described herein can be used to treat a disease or condition caused by or associated with autoimmunity, inflammation, or a lack of blood vessels or need for vascularization. The methods described herein may also include a step of evaluating the symptoms of the disease or condition in a subject prior to treatment with or administration of the lysates, total protein products, or compositions described herein. The subject can then be evaluated using the same diagnostic tests after administration of the lysates, total protein products, or compositions to determine whether the subject's condition has improved.

Treatment may include administration of a lysate, total protein product, or a composition containing a lysate in various unit doses. Each unit dose will ordinarily contain a predetermined quantity of a lysate or total protein product described herein. The quantity to be administered, and the particular route of administration and formulation are within the skill of those in the clinical arts. A unit dose need not be administered as a single injection but may comprise continuous infusion over a set period of time. Dosing may be performed using a syringe. The amount of lysate, total protein product, or composition administered may vary depending on the size of the affected area (e.g., the region of the body afflicted with inflammation or a lack of blood vessels). The lysate, total protein product, or lysate- or total protein product-containing composition can be administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). Lysates, total protein products, or compositions can be administered in two or more doses (e.g., two, three, four, five, or more different doses) or at the same dose two or more times (e.g., two, three, four, five, six, or more times over the course of an hour, day, week, month, or year). The lysates or total protein products may also be diluted prior to administration or formulation in a composition (e.g., diluted by 1:2, 1:5, 1:10, 1:20, 1:50, 1:100, 1:250, or more).

The lysates, total protein products, and compositions described herein are administered in an amount sufficient to reduce inflammation, reduce pain, swelling, and/or stiffness associated with inflammation or an autoimmune or inflammatory disease or condition (e.g., joint pain or stiffness associated with scleroderma or arthritis, such as rheumatoid arthritis), increase or induce angiogenesis (e.g., increase the number or extent of blood vessels in a tissue), or treat a disease or condition described herein (e.g., an autoimmune or inflammatory disease or condition, an inflammation-related skin disease or condition, bursitis of the hip, knee, elbow, or shoulder; tendon inflammation, e.g., tendinitis, such as tennis elbow; joint inflammation, e.g., arthritis, such as osteoarthritis, rheumatoid arthritis, reactive arthritis, or psoriatic arthritis; jaw inflammation, e.g., TMJ; inflammation of a gland, e.g., a salivary gland, such as in Sjogren's syndrome; back pain; neck pain; sciatica; trigger finger; carpal tunnel syndrome and other entrapment syndromes; synovitis; rotator cuff syndrome; impingement syndrome; alopecia (e.g., alopecia areata); frozen shoulder syndrome; fasciitis; gout; or an angiogenesis-related disease or condition (e.g., a cardiovascular disease, such as ischemia, risk of losing a limb, tissue transplant, abnormal or poor vasculature, or a wound (e.g., for promoting wound healing)). Inflammation, symptoms of inflammation or an autoimmune disease (e.g., pain, swelling, stiffness, cytokine levels, and/or auto-antibody levels), angiogenesis (e.g., vascularization), and the condition of the skin can be evaluated using standard methods known by those of skill in the art and may be improved after administration of the lysates, total protein products, or compositions described herein compared to inflammation, symptoms of inflammation (e.g., pain, swelling, stiffness, cytokine levels, and/or autoantibody levels), angiogenesis (e.g., vascularization), and the condition of the skin prior to treatment or compared to untreated subjects with the same disease or condition. Symptoms of diseases and conditions described herein can be evaluated using standard methods known to those of skill in the art and may be reduced (e.g., the subject's condition may be improved) after administration of the lysates, total protein products, or compositions described herein compared to symptoms prior to administration of the lysates, total protein products, or compositions described herein or compared to untreated subjects with the same disease or condition. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the lysate, total protein product, or composition depending on the dose and route of administration used for treatment. Depending on the outcome of the evaluation, the patient may receive additional treatments.

Use of Perivascular Tissue Lysates, MSC Total Protein Products, and/or MSC Lysates for Skincare

Cosmetic Compositions

Lysates prepared from umbilical cord perivascular tissue contain nearly 200 proteins that may be useful for skincare. Therefore, perivascular tissue lysates, MSC total protein products (e.g., HTPPs), and/or MSC (e.g., HUCPVC) lysates can be incorporated into a variety of products for cosmetic use. Lysates prepared using perivascular tissue and/or MSCs (e.g., HUCPVCs) and MSC total protein products (e.g., HTPPs) may be incorporated into cosmetic compositions.

Perivascular tissue lysates, MSC total protein products (e.g., HTPPs), and/or MSC (e.g., HUCPVC) lysates can be incorporated into skincare products for topical application, such as a face or body cream (e.g., a moisturizer, e.g., a facial moisturizer, body moisturizer, foot moisturizer, or a hand moisturizer, or an under-eye cream, foot cream, or hand cream), lotion (e.g., face lotion, body lotion, foot lotion, or hand lotion), ointment, oil (e.g., face oil, body oil, or hair oil), serum, essence, gel (e.g., a gel moisturizer or an under-eye gel), mist (e.g., a skincare product that is sprayed onto the skin), or mask (e.g., a sheet mask, cream mask, clay mask, gel mask, overnight mask (e.g., a sleeping mask), exfoliating mask, or enzyme mask). Perivascular tissue lysates, MSC total protein products (e.g., HTPPs), and/or MSC (e.g., HUCPVC) lysates can also be incorporated into make-up products such as foundation (e.g., liquid foundation, cream foundation, or power foundation), blush (e.g., liquid blush, cream blush, or powder blush), eyeshadow (e.g., liquid eyeshadow, cream eyeshadow, or powder eyeshadow), mascara, eyeliner, lip products (e.g., lipstick, lip gloss, lip balm, lip pencils), setting powders, setting sprays, tinted moisturizers, BB creams, CC creams, primers, tinted under-eye creams, concealers, or nail products (e.g., nail polish, nail polish remover, nail hardeners, or cuticle removers). Light-protective products, such as sunscreens (e.g., mineral or chemical sunscreens in lotion, cream, oil, spray, or milk form) and after sun products (e.g., lotions and gels, such as aloe-containing lotions and gels), can also include perivascular lysates, MSC total protein products (e.g., HTPPs), and/or MSC (e.g., HUCPVC) lysates. Perivascular lysates, MSC total protein products (e.g., HTPPs), and/or MSC (e.g., HUCPVC) lysates can also be incorporated into skin cleansing products (e.g., liquid or bar soaps, detergents, body wash, or face wash), bath products (e.g., bath milk, bath cubes, bath salts, bubble bath), skin-tanning products (e.g., tanning creams, lotions, or oils), deodorants (e.g., solid deodorant, gel deodorant, spray deodorant, or deodorant paste), antiperspirants (e.g., solid antiperspirant, spray antiperspirant, gel antiperspirant, or paste antiperspirant), hair removal products (e.g., depilation products, such as cream, paste, liquid, gel, foam, or powder-based hair removing products), shaving products (e.g., shave soap, shave cream, shave gels, shave foams, pre-shaving products, aftershave, or aftershave lotion), fragrances (e.g., solid fragrances, liquid (e.g., spray or rollerball) fragrances, oil fragrances, or cream fragrances), or insect repellents (e.g., insect repellant lotions, sprays, sticks, or oils). Hair care products can also be prepared to contain perivascular lysates, MSC total protein products (e.g., HTPPs), and/or MSC (e.g., HUCPVC) lysates, such as hair styling products (e.g., styling creams, gels, foams, or sprays), shampoos (e.g., liquid shampoo or dry shampoo), conditioners, pomades, hair rinses, pre-treatment preparations, hair tonics, hair oils, detanglers, hair-waving products, intensive hair treatments, hair treatment packs, hair bleaching products (e.g., hydrogen peroxide solutions, lightening shampoos, bleaching creams, bleaching powders, bleaching pastes, or bleaching oils), and hair coloring products (e.g., temporary, semi-permanent or permanent hair colorants, products containing self-oxidizing dyes, or natural hair colorants, such as henna or chamomile).

Lysate- and total protein product-containing cosmetic compositions may be used to target a number of skin concerns, such as redness, dryness, fine lines, uneven skin tone (e.g., hyperpigmentation), uneven skin texture, acne, dullness, scarring, stretch marks, and pore size. The cosmetic compositions may have anti-aging effects (e.g., reduce the appearance of fine lines and wrinkles, reduce the appearance of age spots, and/or firm or tighten the skin), brightening effects, reduce redness, hydrate the skin, increase collagen production, stop collagen degradation, reduce skin irritation (e.g., skin irritation caused by a hair removal product (e.g., a chemical depilation product), by an abrasive scrub, by a peel, or shaving, or by waxing), reduce the appearance of scars or pockmarks, reduce the appearance of stretch marks, soothe sunburned skin, and/or reduce the appearance of hyperpigmentation. Cosmetic compositions containing perivascular tissue lysates, MSC total protein products (e.g., HTPPs), and/or MSC (e.g., HUCPVC) lysates may be topically applied to the face, scalp, hair, or body (e.g., hands, feet, neck, torso, chest, back, legs, under-arms, or nails).

Perivascular tissue lysates, MSC total protein products (e.g., HTPPs), and/or MSC (e.g., HUCPVC) lysates can be incorporated into cosmetic compositions that contain other active ingredients, such as tocopherol, tocopherol acetate, tocopherol palmitate, deoxyribonucleic acid, retinol, bisabolol, allantoin, phytantriol, panthenol, amino acids, essential oils, plant extracts, vitamin complexes, retinoids (e.g., retinol), Vitamin C, Vitamin A, acids (e.g., alpha-hydroxy acids, beta-hydroxy acid, glycolic acid, kojic acid, ascorbic acid (e.g., L-ascorbic acid), hyaluronic acid, or alpha-lipolic acid), hydroquinone, copper peptide, Vitamin E, dimethylaminoethanol (DMAE), niacinamide, ceramides, pseudoceramides, curcuminoids (e.g., turmeric), and antioxidants (e.g., squalene). These products do not require a prescription and can be obtained at a drugstore or a retailer that sells skincare products. The cosmetic compositions may be formulated with additional compounds typically included in cosmetics, such as fatty alcohols, esters of fatty acids, natural or synthetic triglycerides, pearlescent waxes, hydrocarbon oils, silicones or siloxanes, fluorinated or perfluorinated oils, emulsifiers, superfatting agents, surfactants, consistency regulators/thickeners, theology modifiers, polymers, deodorizing active ingredients, anti-dandruff agents, film formers, hydrotropic agents, preservatives, bacteria inhibiting agents, perfume oils, colorants, polymeric beads, hollow spheres, solubilizers, structurants, opacifiers, complexing agents (e.g., EDTA), or insect repellents.

The lysates, total protein products, and cosmetic compositions described herein may be administered to a subject in need thereof (e.g., a subject with a skin concern) by topical administration to the skin (e.g., to an area of the skin associated with a skin concern, e.g., redness, dryness, fine lines, hyperpigmentation, irritation, scars or pockmarks, stretch marks, or sunburn. Lysates and cosmetic compositions that are administered topically may be administered once a week, twice a week, three times a week, four times a week, five times a week, six times a week, daily, twice daily, or more frequently. Subjects that may be treated as described herein are subjects having a skin concern. The methods described herein may also include a step of evaluating the condition of the subject's skin prior to administration of the lysates, total protein products, or cosmetic compositions described herein. The same evaluation can then be performed after administration of the lysates, total protein products, or cosmetic compositions to determine whether the condition of the subject's skin has improved. The amount of lysate, total protein product, or cosmetic composition administered may vary depending on the size of the affected area (e.g., the area of skin associated with the skin concern). The lysate or total protein product may be included in the cosmetic composition in a therapeutically effective amount, such as from 0.01% (w/w) to 100% (w/w) (e.g., 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 25% (w/w), 30% (w/w), 40% (w/w), 50% (w/w) 60% (w/w), 70% (w/w), 80% (w/w), 90% (w/w), or 100% (w/w)). The lysates or total protein products may also be diluted prior to administration or formulation in a cosmetic composition (e.g., diluted by 1:2, 1:5, 1:10, 1:20, 1:50, 1:100, 1:250, or more).

The lysates, total protein products, and cosmetic compositions described herein are administered in an amount sufficient to improve the condition of the skin (e.g., reduce skin dryness, reduce the appearance of fine lines and wrinkles, reduce uneven skin tone, reduce skin redness, reduce the appearance of dark spots, reduce hyperpigmentation, improve skin texture, reduce acne, reduce skin dullness, reduce pore size, firm or tighten the skin, reduce the appearance of age spots, improve skin brightness, improve skin hydration, reduce the appearance of scars or pockmarks, reduce the appearance of stretch marks, soothe sunburn, reduce skin irritation, increase collagen production, and/or reduce or inhibit collagen degradation). The condition of the skin can be evaluated using standard methods (e.g., visual inspection) and may be improved after administration of the lysates, total protein products, or cosmetic compositions described herein compared to the condition of the skin prior to treatment or compared to untreated subjects with the same skin concern. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the lysates or cosmetic compositions described herein. The subject may be evaluated 1 month, 2 months, 3 months, 4 months, 5 months, 6 months or more following administration of the lysate or cosmetic composition. Depending on the outcome of the evaluation, the subject may continue to apply the lysate, total protein product, or cosmetic composition.

Dermatologic Compositions

Perivascular tissue lysates, MSC total protein products (e.g., HTPPs), and/or MSC (e.g., HUCPVC) lysates can also be incorporated into topical dermatologic compositions (e.g., medications prescribed by a doctor, such as a dermatologist). Lysates and total protein products prepared from MSCs (e.g., HUCPVCs), such as cultured MSCs and umbilical cord perivascular tissue, can be used to make dermatologic compositions, such as a face or body cream, lotion, ointment, gel, or spray. The dermatologic composition made with a perivascular tissue lysate, MSC total protein product (e.g., HTPP), and/or MSC (e.g., HUCPVC) lysate can be used to treat redness, irritation, acne, dryness, fine lines and wrinkles, uneven skin tone (e.g., hyperpigmentation), uneven skin texture, scarring or pockmarks, hair loss, itchiness, rashes, and/or an auto-immune or inflammation-related skin condition such as dermatitis (e.g., atopic dermatitis, also known as eczema), psoriasis, scleroderma, dermatomyositis, epidermolysis bullosa, pemphigus vulgaris, or alopecia (e.g., alopecia areata). Dermatologic compositions containing perivascular tissue lysates, MSC total protein products (e.g., HTPPs), or cultured MSC (e.g., HUCPVC) lysates may be topically applied to the skin of the face, scalp, or body (e.g., hands, feet, neck, torso, chest, back, or legs).

Perivascular tissue lysates, MSC total protein products (e.g., HTPPs), and/or MSC (e.g., HUCPVC) lysates can be included in a dermatologic product along with other agents that are typically prescribed for topical application to treat skin conditions or diseases, such as steroids and corticosteroids (e.g., clobetasol propionate, halobetasol propionate, fluocinonide, diflorasone, desoximetasone, clocortolone pivalate, mometasone furoate, triamcinolone acetonide, betamethasone valerate, fluocinolone acetonide, fluticasone propionate, prednicarvate, hydrocortisone probutate, alcometasone diproppionate, desonide, flurandrenolide, fluticasone, hydrocortisone valerate, hydrocortisone butyrate, and hydrocortisone), coal tar, retinoids (e.g., tretinoin, adapalene, and tazarotene), calcineurin inhibitors (e.g., tacrolimus, pimecrolimus), vitamin D analogs, doxepin, benzoyl peroxide, azeliac acid, dapsone, phosphodiesterase-4 inhibitors (e.g., crisaborole), anthralin, anti-bacterial agents (e.g., mupirocin, clindamycin, or erythromycin), and anti-fungal agents (e.g., clotrimazole, ketoconazole, or terbinafine). Dermatologic compositions made with perivascular tissue lysates, MSC total protein products (e.g., HTPPs), and/or MSC (e.g., HUCPVC) lysates can be obtained from a pharmacy with a prescription from a doctor and may be prescribed to treat a specific disease or skin condition.

The dermatologic compositions described herein may be topically administered to a subject in need thereof (e.g., a subject with a skincare concern or an auto-immune or inflammation-related skin condition, such as dermatitis (e.g., atopic dermatitis, also known as eczema), psoriasis, scleroderma, dermatomyositis, epidermolysis bullosa, pemphigus vulgaris, or alopecia (e.g., alopecia areata)). The dermatologic composition may be administered (e.g., applied) to an area of the skin associated with a skin concern (e.g., redness, dryness, irritation, acne, fine lines or wrinkles, hyperpigmentation, scarring or pockmarks, hair loss, itchiness, or a rash), or a skin condition (e.g., psoriasis, atopic dermatitis, or alopecia). Dermatologic compositions may be administered once, or more than once (e.g., once annually, twice annually, three times annually, bi-monthly, monthly, or weekly). In some embodiments, the dermatologic composition is administered once a week, twice a week, three times a week, four times a week, five times a week, six times a week, daily, twice daily, or more frequently. Subjects that may be treated as described herein are subjects having a skin concern or an auto-immune or inflammation-related skin condition described herein (e.g., dermatitis (e.g., atopic dermatitis, also known as eczema), psoriasis, scleroderma, dermatomyositis, epidermolysis bullosa, pemphigus vulgaris, or alopecia (e.g., alopecia areata)). The methods described herein may also include a step of evaluating the condition of the subject's skin prior to administration of the dermatologic compositions described herein. The same evaluation can then be performed after administration of the dermatologic compositions to determine whether the condition of the subject's skin has improved. Treatment may include administration of lysates or a composition containing a lysate in various unit doses. The amount of dermatologic composition administered may vary depending on the size of the affected area (e.g., the area of skin afflicted with the disease or condition). The lysate or total protein product may be included in the dermatological composition in a therapeutically effective amount, such as from 0.01% (w/w) to 20% (w/w) (e.g., 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), or 20% (w/w)). The lysates or total protein products may also be diluted prior to administration or formulation in a dermatologic composition (e.g., diluted by 1:2, 1:5, 1:10, 1:20, 1:50, 1:100, 1:250, or more). Dermatologic compositions can be administered in two or more doses (e.g., two, three, four, five, or more different doses, e.g., depending on the severity of the skin condition in various regions of the body or as the skin condition improves) or at the same dose two or more times (e.g., two, three, four, five, six, or more times over the course of an hour, day, week, month, or year).

The dermatologic compositions described herein are administered in an amount sufficient to reduce itchiness of the skin, treat a rash, improve the condition of the skin (e.g., reduce skin dryness, reduce the appearance of fine lines and wrinkles, reduce uneven skin tone, reduce the appearance of dark spots, reduce hyperpigmentation, improve skin texture, reduce acne, improve skin hydration, reduce skin irritation, reduce the appearance of scarring or pockmarks, reduce hair loss, increase hair growth), or treat a skin condition described herein (e.g., dermatitis (e.g., atopic dermatitis, also known as eczema), psoriasis, scleroderma, dermatomyositis, epidermolysis bullosa, pemphigus vulgaris, or alopecia (e.g., alopecia areata)). The condition of the skin can be evaluated using standard methods known by those of skill in the art and may be improved after administration of the dermatologic compositions described herein compared to the condition of the skin prior to treatment or compared to untreated subjects with the same skin condition or concern. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the dermatologic compositions described herein. The patient may be evaluated 1 month, 2 months, 3 months, 4 months, 5 months, 6 months or more following administration of the dermatologic composition depending on the dose and frequency of application used for treatment. Depending on the outcome of the evaluation, the subject may continue to apply the dermatologic composition or be prescribed a different dose of the dermatologic composition.

Use of Perivascular Stem Cell, MSC Total Protein Products, and/or Tissue Lysates as Cell Culture Reagents

Perivascular tissue lysates, MSC total protein products (e.g., HTPPs), and/or MSC (e.g., HUCPVC) lysates can also be used as cell culture reagents to promote or improve the health, proliferation, and/or longevity of cultured cells. Lysates prepared using perivascular tissue and/or uMSCs (e.g., HUCPVCs) and MSC total protein products prepared using MSCs (e.g., HUCPVCs), may be applied to cultured cells. To produce lysates or total protein products for cell culture, a lysate or total protein product can be added to a cell culture medium. The cell culture medium can be selected based on the type of cell to be cultured with the lysate or total protein product. The concentration of the lysate or total protein product in the cell culture medium may be from 0.01% (w/w) to 20% (w/w) (e.g., 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), or 20% (w/w)). The lysate or total protein product may be added to directly to cultured cells or incorporated into a cell culture product along with other agents typically added to cultured cells.

Cells that may be treated with the perivascular tissue lysates, MSC total protein products (e.g., HTPPs), and/or MSC (e.g., HUCPVC) lysates described herein include perivascular stromal cells, MSCs, embryonic stem cells, neurons, glial cells, induced pluripotent stem cells, a hematopoietic stem cell, an endothelial stem cell, an epithelial stem cell, an adipose stem or progenitor cell, a germline stem cell, a lung stem or progenitor cell, a mammary stem cell, an olfactory adult stem cell, a hair follicle stem cell, an intestinal stem or progenitor cell, a multipotent stem cell, an amniotic stem cell, a cord blood stem cell, a neural stem or progenitor cell, an adult stem cell, a somatic stem cell, a tissue-specific stem cell, a totipotent stem cell, a fibroblast, an immune cell, a germ cell, a somatic cell, an exocrine cell, a pancreatic progenitor, an endocrine progenitor, a hepatoblast, a myoblast, a preadipocyte, a hepatocyte, a chondrocyte, a smooth muscle cell, a cancer cell, a bone cell, a synovial cell, a tendon cell, a ligament cell, a meniscus cell, an adipose cell, a skeletal muscle cell, a cardiac muscle cell, an islet beta-cell, a cardiomyocyte, a blood cell, an exocrine progenitor, a ductal cell, an acinar cell, an alpha cell, a beta cell, a delta cell, a PP cell, a cholangiocyte, a white or brown adipocyte, a hormone-secreting cell, an epidermal keratinocyte, an epithelial cell, a kidney cell, a skeletal joint synovium cell, a periosteum cell, a perichondrium cell, a cartilage cell, an endothelial cell, a pericardium cell, a meningeal cell, a keratinocyte precursor cell, a keratinocyte stem cell, a pericyte, an ependymal cell, a cell isolated from an amniotic or placental membrane, a serosal cell, a cell derived from skin, heart, brain or spinal cord, liver, lung, kidney, pancreas, bladder, bone marrow, spleen, intestine, or stomach, or an immortalized cell or cell line.

Pharmaceutical Compositions

The lysates and/or or total protein products described herein may be incorporated into a composition containing a vehicle or excipient for administration to a patient, such as a human patient suffering from an autoimmune or inflammatory disease or condition, an inflammation-related skin disease or condition, local inflammation, or an angiogenesis-related disease or condition described herein. Pharmaceutical compositions containing lysates and/or total protein products can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of aqueous solutions, e.g., solutions that can be formulated for injection, inhalation (e.g., nebulization or delivery as an aerosol), or topical administration.

The lysates and/or or total protein products described herein can be administered in any physiologically compatible carrier, such as a buffered saline solution. Pharmaceutically acceptable carriers and diluents include saline, aqueous buffer solutions, solvents and/or dispersion media. The use of such carriers and diluents is well known in the art. Other examples include liquid media, for example, Dulbecco's modified eagle's medium (DMEM), sterile saline, sterile phosphate buffered saline, Leibovitz's medium (L15, Invitrogen, Carlsbad, Calif.), dextrose in sterile water, and any other physiologically acceptable liquid. Dispersions may also be prepared in glycerol, liquid polyethylene glycols, and mixtures thereof and in oils. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In many cases, it will be preferable to include isotonic agents, for example, sugars or sodium chloride. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the cloaked cells as described herein.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. The person responsible for administration will, in any event, determine the appropriate dose for the individual subject. Moreover, for human administration, preparations may meet sterility, pyrogenicity, general safety, and purity standards as required by FDA Office of Biologics standards.

Cosmetic or Dermatologic Compositions

The lysates and/or or total protein products described herein may be incorporated into a composition designed for topical application, such as a cosmetic or dermatologic composition. Such compositions may include creams, pastes, gels, ointments, lotions, oils, essences, serums, sprays (e.g., mists), and masks, as described above. These compositions may include one or more additional compounds as described below.

Fatty Alcohols

The composition may include Guerbet alcohols based on fatty alcohols having from 6 to 18, preferably from 8 to 10, carbon atoms, such as cetyl alcohol, stearyl alcohol, cetearyl alcohol, oleyl alcohol, octyldodecanol, benzoates of C₁₂-C₁₅ alcohols, and acetylated lanolin alcohol.

Esters of Fatty Acids

The composition can include esters of linear C₆-C₂₄ fatty acids with linear C₃-C₂₄ alcohols, esters of branched C₆-C₁₃carboxylic acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, and technical-grade mixtures thereof (obtained, for example, in the pressure removal of natural fats and oils, in the reduction of aldehydes from Roelen's oxosynthesis or in the dimerisation of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol, and brassidyl alcohol, and technical-grade mixtures thereof (obtained, for example, in the high-pressure hydrogenation of technical-grade methyl esters based on fats and oils or aldehydes from Roelen's oxosynthesis and as monomer fractions in the dimerisation of unsaturated fatty alcohols).

Examples of ester oils include isopropyl myristate, isopropyl palmitate, isopropyl stearate, isopropyl isostearate, isopropyl oleate, n-butyl stearate, n-hexyl laurate, n-decyl oleate, isooctyl stearate, isononyl stearate, isononyl isononanoate, 2-ethylhexyl palmitate, 2-hexyl laurate, 2-hexyldecyl stearate, 2-octyldodecyl palmitate, oleyl oleate, oleyl erucate, erucyl oleate, erucyl erucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, and octyl hydroxystearate.

The compositions may also contain diethylhexyl 2,6-naphthalate, di-n-butyl adipate, di(2-ethylhexyl) adipate, di(2-ethylhexyl) succinate and diisotridecyl acetate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. The compositions can also include esters of C₆-C₂₄ fatty alcohols and/or Guerbet alcohols with aromatic carboxylic acids, saturated and/or unsaturated, especially benzoic acid, esters of C₂-C₁₂ dicarboxylic acids with linear or branched alcohols having from 1 to 22 carbon atoms or polyols having from 2 to 10 carbon atoms and from 2 to 6 hydroxy groups, or iminodisuccinic acid and iminodisuccinic acid salts [CAS 7408-20-0], or latex particles.

Natural or Synthetic Triglycerides, Including Glyceryl Esters and Derivatives

The composition may include di- or tri-glycerides, based on C₆-C₁₈ fatty acids, modified by reaction with other alcohols (caprylic/capric triglyceride, wheatgerm glycerides, etc.). The composition can include fatty acid esters of polyglycerol (polyglyceryl-n such as polyglyceryl-4 caprate, polyglyceryl-2 isostearate, etc.) or castor oil, hydrogenated vegetable oil, sweet almond oil, wheatgerm oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, and borage oil.

The composition may include waxes, including esters of long-chain acids and alcohols as well as compounds having wax-like properties, e.g. carnauba wax, beeswax (white or yellow), lanolin wax, candelilla wax, ozokerite, japan wax, paraffin wax, microcrystalline wax, ceresin, cetearyl ester wax, and synthetic beeswax. The wax may be a hydrophilic wax, such as cetearyl alcohol or partial glycerides.

Pearlescent Waxes

The composition can include alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially laurone and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

Hydrocarbon Oils

The composition may include mineral oil (light or heavy), petrolatum (yellow or white), microcrystalline wax, paraffinic and isoparaffinic compounds, hydrogenated isoparaffinic molecules such as polydecenes and polybutene, hydrogenated polyisobutene, squalane, isohexadecane, isododecane, and others of vegetable or animal origin.

Silicones or Siloxanes (Organo-Substituted Polysiloxanes)

The composition can include dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form. The composition may include linear polysiloxanes, dimethicone (Dow Corning 200 fluid, Rhodia Mirasil DM), dimethiconol, cyclic silicone fluids, cyclopentasiloxane volatiles (Dow Corning 345 fluid), or phenyltrimethicone (Dow Corning 556 fluid). Also suitable for use in the composition are simethicones, which are mixtures of dimethicones having an average chain length of from 200 to 300 dimethylsiloxane units with hydrogenated silicates. A detailed survey by Todd et al. of suitable volatile silicones may in addition be found in Cosm. Toil. 91, 27 (1976).

Fluorinated or Perfluorinated Oils

The composition may include a fluorinated or perfluorinated oil, such as perfluorohexane, dimethylcyclohexane, ethylcyclopentane, or polyperfluoromethylisopropyl ether.

Emulsifiers

Any conventionally usable emulsifier can be used in the compositions. Emulsifier systems may include, for example: carboxylic acids and their salts: alkaline soaps of sodium, potassium and ammonium, metallic soaps of calcium or magnesium, and organic-based soaps such as lauric, palmitic, stearic and oleic acid. The composition can include alkyl phosphates or phosphoric acid esters, acid phosphates, diethanolamine phosphate, or potassium cetyl phosphate. The composition may include ethoxylated carboxylic acids or polyethylene glycol esters or PEG-n acylates. The composition can include linear fatty alcohols having from 8 to 22 carbon atoms, branched, from 2 to 30 mol of ethylene oxide and/or from 0 to 5 mol of propylene oxide with fatty acids having from 12 to 22 carbon atoms and with alkylphenols having from 8 to 15 carbon atoms in the alkyl group. The composition may include fatty alcohol polyglycol ethers such as laureth-n, ceteareth-n, steareth-n, and oleth-n. The composition can include fatty acid polyglycol ethers such as PEG-n-stearate, PEG-n-oleate, and PEG-n-cocoate. The composition may include monoglycerides or polyol esters. The composition can include C₁₂-C₂₂ fatty acid mono- and di-esters of addition products of from 1 to 30 mol of ethylene oxide with polyols. Fatty acid and polyglycerol esters such as glycerol monostearate, diisostearoyl polyglyceryl-3-diisostearates, polyglyceryl-3-diisostearates, triglyceryl diisostearates, polyglyceryl-2-sesquiisostearates, or polyglyceryl dimerates. Mixtures of compounds from a plurality of those substance classes are also suitable. The composition can include fatty acid polyglycol esters such as diethylene glycol monostearate, fatty acid and polyethylene glycol esters, fatty acid and saccharose esters such as sucrose esters, glycerol and saccharose esters such as sucrose glycerides. The composition may include sorbitol and sorbitan, sorbitan mono- and di-esters of saturated and unsaturated fatty acids having from 6 to 22 carbon atoms and ethylene oxide addition products. The composition can include polysorbate-n series, sorbitan esters such as sesquiisostearate, sorbitan, PEG-(6)-sorbitan isostearate, PEG-(10)-sorbitan laurate, or PEG-17-sorbitan dioleate. The composition can include glucose derivatives, C₈-C₂₂ alkyl-mono and oligo-glycosides and ethoxylated analogues with glucose being preferred as the sugar component. The composition can include OM/emulsifiers such as methyl gluceth-20 sesquistearate, sorbitan stearate/sucrose cocoate, methyl glucose sesquistearate, and cetearyl alcohol/cetearyl glucoside. The composition can include MO emulsifiers such as methyl glucose dioleate/methyl glucose isostearate. The composition can include sulfates and sulfonated derivatives, dialkylsulfosuccinates, dioctyl succinate, alkyl lauryl sulfonate, linear sulfonated paraffins, sulfonated tetrapropylene sulfonate, sodium lauryl sulfates, ammonium and ethanolamine lauryl sulfates, lauryl ether sulfates, sodium laureth sulfates, sulfosuccinates, acetyl isothionates, alkanolamide sulfates, taurines, methyl taurines, or imidazole sulfates. The composition can include amine derivatives, amine salts, ethoxylated amines, and oxiy amines with chains containing a heterocycle, such as alkyl imidazolines, pyridine derivatives, isoquinolines, cetylpyridinium chloride, cetylpyridinium bromide, quaternary ammonium such as cetyltrimethylammonium bromide (CTBA), and stearylalkonium. The composition can include amide derivatives, alkanolamides such as acylamide DEA, ethoxylated amides such as PEG-n acylamide, and oxydiamides. The composition can include polysiloxane/polyalkyl/polyether copolymers and derivatives, dimethicone, copolyols, silicone polyethylene oxide copolymer, or silicone glycol copolymer. The composition can include propoxylated or POE-n ethers (Meroxapols), or Polaxamers or poly(oxyethylene)m-block-poly(oxypropylene)n-block(oxyethylene). The composition can include zwitterionic surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate, and 2-alkyl-3-carboxymethyl-3-hydroxyethylimidazolines each having from 8 to 18 carbon atoms in the alkyl or acyl group, and also cocoacylaminoethylhydroxyethylcarboxymethylglycinate, N-alkylbetaine, and N-alkylaminobetaines. The composition can include alkylimidazolines, alkyl peptides, lipoamino acids, self-emulsifying bases, and the compounds as described in K. F. DePolo, A short textbook of cosmetology, Chapter 8, Table 8-7, p250-251.

The composition can include non-ionic emulsifiers such as PEG-6 beeswax and PEG-6 stearate and polyglyceryl 2-isostearate [Apifac], glyceryl stearate and PEG-100 stearate. [Arlacel 165], PEG-5 glyceryl stearate [Arlatone 983 S], sorbitan oleate (and) polyglyceryl-3 ricinoleate [Arlacel 1689], sorbitan stearate and sucrose cocoate [Arlatone 2121], glyceryl stearate and laureth-23 [Cerasynth 945], cetearyl alcohol and Ceteth-20 [cetomacrogol wax], cetearyl alcohol and polysorbate 60 and PEG-150 and stearate-20 [polawax GP 200, polawax NF], cetearyl alcohol and cetearyl polyglucoside [Emulgade PL 1618], cetearyl alcohol and ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and steareth-7 and steareth-10 [Emulgator E 2155], cetearyl alcohol and steareth-7 and steareth-10 [emulsifying wax U.S.N.F], glyceryl stearate and PEG-75 stearate [Gelot 64], propylene glycol ceteth-3 acetate [Hetester PCS], propylene glycol isoceth-3 acetate [Hetester PHA], cetearyl alcohol and ceteth-12 and oleth-12 [Lanbritol Wax N21], PEG-6 stearate and PEG-32 stearate [Tefose 1500], PEG-6 stearate and ceteth-20 and steareth-20 [Tefose 2000], PEG-6 stearate and ceteth-20 and glyceryl stearate and steareth-20 [Tefose 2561], and glyceryl stearate and ceteareth-20 [Teginacid H, C, X].

The composition can include anionic emulsifiers such as PEG-2 stearate SE, glyceryl stearate SE [Monelgine, Cutina KD], propylene glycol stearate [Tegin P], cetearyl alcohol and sodium cetearyl sulfate [Lanette N, Cutina LE, Crodacol GP], cetearyl alcohol and sodium lauryl sulfate [Lanette W], trilaneth-4 phosphate and glycol stearate and PEG-2 stearate [Sedefos 75], and glyceryl stearate and sodium lauryl sulfate [Teginacid Special]. The composition may include cationic acid bases such as cetearyl alcohol and cetrimonium bromide.

The emulsifiers may be used in an amount of, for example, from 1 to 30% by weight, especially from 4 to 20% by weight and preferably from 5 to 10% by weight, based on the total weight of the composition. When formulated in O/W emulsions, the preferred amount of such emulsifier systems may constitute 5% to 20% of the oil phase.

Superfatting Agents

Substances suitable for use as superfatting agents are, for example, lanolin and lecithin, and also polyethoxylated or acrylated lanolin and lecithin derivatives, polyol fatty acid esters, monoglycerides, and fatty acid alkanolamides, the latter simultaneously acting as foam stabilizers.

Surfactants

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isothionates, fatty acid sarcosinates, fatty acid taurides, fatty acid glutamates, α-olefin sulfonates, ethercarboxylic acids, alkyl oligoglucosides, fatty acid glucamides, alkylamidobetaines and/or protein fatty acid condensation products, the latter preferably being based on wheat proteins.

Consistency Regulators/Thickeners and Theology Modifiers

The composition can include silicon dioxide, magnesium silicates, aluminum silicates, polysaccharides, or derivatives thereof for example hyaluronic acid, xanthan gum, guar-guar, agar-agar, alginates, carragheenan, gellan, pectins, or modified cellulose such as hydroxycellulose or hydroxypropyl methylcellulose. The composition may include polyacrylates or homopolymers of crosslinked acrylic acids and polyacrylamides, carbomers (Carbopol types 980, 981, 1382, ETD 2001, ETD2020, Ultrez 10), or the Salcare range such as Salcare SC80 (steareth-10 allyl ether/acrylate copolymer), Salcare SC81 (acrylate copolymer), Salcare SC91, and Salcare AST (sodium acrylate copolymer/PPG-1 trideceth-6), Sepigel 305 (polyacrylamide/laureth-7), Simulgel NS and Simulgel EG (hydroxyethyl acrylate/sodium acryloyldimethyl taurate copolymer), Stabilen 30 (acrylate/vinyl isodecanoate crosspolymer), Pemulen TR-1 (acrylate/₁₀-C₃₀ alkyl acrylate crosspolymer), Luvigel EM (sodium acrylate copolymer), or Aculyn 28 (acrylate/beheneth-25 methacrylate copolymer).

Polymers

Suitable cationic polymers for use in the compositions of the invention are, for example, cationic cellulose derivatives, for example a quaternised hydroxymethyl cellulose obtainable under the name Polymer JR 400 from Amerchol, cationic starches, copolymers of diallylammonium salts and acrylamides, quaternised vinylpyrrolidone/vinyl imidazole polymers, for example Luviquat® (BASF), condensation products of polyglycols and amines, quaternised collagen polypeptides, for example lauryldimonium hydroxypropyl hydrolysed collagen (Lamequat®L/Grünau), quaternised wheat polypeptides, polyethyleneimine, cationic silicone polymers, for example amidomethicones, copolymers of adipic acid and dimethylaminohydroxypropyl diethylenetriamine (Cartaretin/Sandoz), copolymers of acrylic acid with dimethyldiallylammonium chloride (Merquat 550/Chemviron), polyaminopolyamides, as described, for example, in FR-A-2 252 840, and the crosslinked water-soluble polymers thereof, cationic chitin derivatives, for example of quaternised chitosan, optionally distributed as microcrystals; condensation products of dihaloalkyls, for example dibromobutane, with bisdialkylamines, for example bisdimethylamino-1,3-propane, cationic guar gum, for example Jaguar C-17, Jaguar C-16 from Celanese, and quaternised ammonium salt polymers, for example Mirapol A-15, Mirapol AD-1, and Mirapol AZ-1 from Miranol. As anionic, zwitterionic, amphoteric and non-ionic polymers there come into consideration, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octyl acrylamide/methyl methacrylate-tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatized cellulose ethers and silicones. Furthermore, the polymers as described in EP 1 093 796 (pages 3-8, paragraphs 17-68) may be used.

Deodorizing Active Ingredients

As deodorizing active ingredients there come into consideration, for example, antiperspirants, for example aluminum chlorohydrates (see J. Soc. Cosm. Chem. 24:281, 1973). Under the trade mark Locron® of Hoechst AG, Frankfurt (FRG), there is available commercially, for example, an aluminium chlorohydrate corresponding to formula Al₂(OH)₅Cl×2.5H₂O, the use of which is especially preferred (see J. Pharm. Pharmacol. 26:531, 1975). Besides the chlorohydrates, it is also possible to use aluminium hydroxyacetates and acidic aluminium/zirconium salts. Esterase inhibitors may be added as further deodorizing active ingredients. Such inhibitors are preferably trialkyl citrates, such as trimethyl citrate, tripropyl citrate, triisopropyl citrate, tributyl citrate and especially triethyl citrate (Hydagen CAT, Henkel), which inhibit enzyme activity and hence reduce odor formation. Further substances that come into consideration as esterase inhibitors are sterol sulfates or phosphates, for example lanosterol, cholesterol, campesterol, stigmasterol, and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, and hydroxycarboxylic acids and esters thereof, for example citric acid, malic acid, tartaric acid or tartaric acid diethyl ester. Antibacterial active ingredients that influence the germ flora and kill or inhibit the growth of sweat-decomposing bacteria can likewise be present in the preparations (especially in stick preparations). Examples include chitosan, phenoxyethanol and chlorhexidine gluconate. 5-chloro-2-(2,4-dichlorophenoxy)-phenol (Triclosan, Irgasan, Ciba Specialty Chemicals Inc.) has also proved especially effective.

Anti-Dandruff Agents

The composition may include an anti-dandruff agent, such as climbazole, octopirox, or zinc pyrithione.

Film Formers

Customary film formers include, for example, chitosan, microcrystalline chitosan, quaternised chitosan, polyvinylpyrrolidone, vinylpyrrolidone/vinyl acetate copolymers, polymers of quaternary cellulose derivatives containing a high proportion of acrylic acid, collagen, hyaluronic acid and salts thereof, and similar compounds.

Antioxidants

The composition may include light-protective substances of the antioxidant kind that interrupt the photochemical reaction chain triggered when UV radiation penetrates the skin or hair. Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid, phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl)propionyl]sulfanilic acid (and salts thereof, for example the disodium salts), zinc and derivatives thereof (e.g. ZnO, ZnSO₄), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS (=“Hindered Amine Light Stabilizers”) compounds may also be included. Further synthetic and natural antioxidants are listed e.g. in patent IA/00025731: structures 1-3 (page 2), structure 4 (page 6), structures 5-6 (page 7) and compounds 7-33 (page 8-14).

The amount of antioxidants present is usually from 0.001 to 30% by weight, preferably from 0.01 to 3% by weight.

Hydrotropic Agents

To improve the flow behavior it is also possible to employ hydrotropic agents, for example ethoxylated or non-ethoxylated mono-alcohols, diols, or polyols with a low number of carbon atoms or their ethers (e.g. ethanol, isopropanol, 1,2-dipropanediol, propylene glycol, glycerol, ethylene glycol, ethylene glycol monoethyl ether, ethylene glycol monobutyl ether, propylene glycol monomethyl ether, propylene glycol monoethyl ether, propylene glycol monobutyl ether, diethylene glycol monomethyl ether; diethylene glycol monoethyl ether, diethylene glycol monobutyl ether, and similar products). The polyols that come into consideration for that purpose have preferably from 2 to 15 carbon atoms and at least two hydroxy groups. The polyols may also contain further functional groups, especially amino groups, and/or may be modified with nitrogen. Typical examples are as follows: glycerol, alkylene glycols, for example ethylene glycol, diethylene glycol, propylene glycol, butylene glycol, hexylene glycol and also polyethylene glycols having an average molecular weight of from 100 to 1000 Dalton; technical-grade oligoglycerol mixtures having an intrinsic degree of condensation of from 1.5 to 10, for example technical-grade diglycerol mixtures having a diglycerol content of from 40 to 50% by weight; methylol compounds, such as, especially, trimethylolethane, tri methylolpropane, trimethylolbutane, pentaerythritol and dipentaerythritol; lower alkyl-glucosides, especially those having from 1 to 8 carbon atoms in the alkyl radical, for example methyl and butyl glucoside; sugar alcohols having from 5 to 12 carbon atoms, for example sorbitol or mannitol; sugars having from 5 to 12 carbon atoms, for example glucose or saccharose; amino sugars, for example glucamine; and dialcohol amines, such as diethanolamine or 2-amino-1,3-propanediol.

Preservatives and Bacteria-Inhibiting Agents

The composition may include a preservative, such as methyl, ethyl, propyl and butyl parabens, benzalkonium chloride, 2-bromo-2-nitro-propane-1,3-diol, dehydroacetic acid, diazolidinyl urea, 2-dichloro-benzyl alcohol, DMDM hydantoin, formaldehyde solution, methyldibromoglutanitrile, phenoxyethanol, sodium hydroxymethylglycinate, imidazolidinyl urea, triclosan, and further substance classes listed in the following reference: K. F. DePolo—A short textbook of cosmetology, Chapter 7, Table 7-2, 7-3, 7-4 and 7-5, p210-219.

Bacteria-Inhibiting Agents

Typical examples of bacteria-inhibiting agents are preservatives that have a specific action against gram-positive bacteria, such as 2,4,4′-trichloro-2′-hydroxydiphenyl ether, chlorhexidine (1,6-di(4-chlorophenyl-biguanido)hexane), or TCC (3,4,4′-trichlorocarbanilide). A large number of aromatic substances and ethereal oils also have antimicrobial properties. Typical examples are the active ingredients eugenol, menthol, and thymol in clove oil, mint oil, and thyme oil. A natural deodorising agent of interest is the terpene alcohol farnesol (3,7,11-trimethyl-2,6,10-dodecatrien-1-ol), which is present in lime blossom oil. Glycerol monolaurate has also proved to be a bacteriostatic agent. The amount of the additional bacteria-inhibiting agents present is usually from 0.1 to 2% by weight, based on the solids content of the preparations.

Perfume Oils

The compositions may contain perfume oils, such as mixtures of natural and/or synthetic aromatic substances. Natural aromatic substances are, for example, extracts from blossom (lilies, lavender, roses, jasmine, neroli, ylang-ylang), from stems and leaves (geranium, patchouli, petitgrain), from fruit (aniseed, coriander, carraway, juniper), from fruit peel (bergamot, lemons, oranges), from roots (mace, angelica, celery, cardamom, costus, iris, calmus), from wood (pinewood, sandalwood, guaiacum wood, cedarwood, rosewood), from herbs and grasses (tarragon, lemon grass, sage, thyme), from needles and twigs (spruce, pine, Scots pine, mountain pine), from resins and balsams (galbanum, elemi, benzoin, myrrh, olibanum, opoponax). Animal raw materials also come into consideration, for example civet and castoreum. Typical synthetic aromatic substances are, for example, products of the ester, ether, aldehyde, ketone, alcohol, or hydrocarbon type. Aromatic substance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate, and benzyl salicylate. The ethers include, for example, benzyl ethyl ether; the aldehydes include, for example, the linear alkanals having from 8 to 18 hydrocarbon atoms, citral, citronellal, citronellyl oxyacetaldehyde, cyclamen aldehyde, hydroxycitronellal, filial, and bourgeonal; the ketones include, for example, the ionones, isomethylionone, and methyl cedryl ketone; the alcohols include, for example, anethol, citronellol, eugenol, isoeugenol, geraniol, linalool, phenyl ethyl alcohol, and terpinol; and the hydrocarbons include mainly the terpenes and balsams. It is preferable, however, to use mixtures of various aromatic substances that together produce an attractive scent. Ethereal oils of relatively low volatility, which are chiefly used as aroma components, are also suitable as perfume oils, e.g. sage oil, camomile oil, clove oil, melissa oil, oil of cinnamon leaves, lime blossom oil, juniper berry oil, vetiver oil, olibanum oil, galbanum oil, labdanum oil, and lavandin oil. Preference is given to the use of bergamot oil, dihydromyrcenol, filial, lyral, citronellol, phenyl ethyl alcohol, hexyl cinnamaldehyde, geraniol, benzyl acetone, cyclamen aldehyde, linalool, boisambrene forte, ambroxan, indole, hedione, sandelice, lemon oil, tangerine oil, orange oil, allyl amyl glycolate, cyclovertal, lavandin oil, muscatel sage oil, damascone, bourbon geranium oil, cyclohexyl salicylate, vertofix coeur, iso-E-Super, Fixolide NP, evernyl, iraldein gamma, phenylacetic acid, geranyl acetate, benzyl acetate, rose oxide, romillat, irotyl, and floramat alone or in admixture with one another.

Colorants

The composition may contain colorants that are suitable and permitted for cosmetic purposes, as compiled, for example, in the publication “Kosmetische Farbemittel” of the Farbstoffkommission der Deutschen Forschungsgemeinschaft, Verlag Chemie, Weinheim, 1984, pages 81 to 106. The colorants are usually used in concentrations of from 0.001 to 0.1% by weight, based on the total mixture.

Polymeric Beads or Hollow Spheres as SPF Enhancers

The composition may include SPF enhancers, such as non-active ingredients like styrene/acrylate copolymers, silica beads, spheroidal magnesium silicate, and crosslinked polymethylmethacrylates (PMMA; Micopearl M305 Seppic). Hollow-sphere additives (Sunspheres® ISP, Silica Shells Kobo.) deflect radiation and the effective path length of the photon is therefore increased (EP 0 893 119). Some beads provide a soft feel during spreading. Moreover, the optical activity of such beads, e.g. Micropearl M305, can modulate skin-shine by eliminating reflection phenomena and may indirectly scatter the UV light.

Other Additives

The compositions may also contain antifoams, such as silicones, structurants, such as maleic acid, solubilizers, such as ethylene glycol, propylene glycol, glycerol, or diethylene glycol, opacifiers, such as latex, styrene/PVP, or styrene/acrylamide copolymers, complexing agents, such as EDTA, NTA, alaninediacetic acid, or phosphonic acids, propellants, such as propane/butane mixtures, N₂O, dimethyl ether, CO₂, N₂, or air, so-called coupler and developer components as oxidation dye precursors, reducing agents, such as thioglycolic acid and derivatives thereof, thiolactic acid, cysteamine, thiomalic acid, or mercaptoethanesulfonic acid, or oxidizing agents, such as hydrogen peroxide, potassium bromate or sodium bromate.

Suitable insect repellents for use in the compositions described herein include N,N-diethyl-m-toluamide, 1,2-pentanediol, or insect repellent 3535. Suitable self-tanning agents for use in the compositions described herein include dihydroxyacetone and/or erythrulose or dihydroxy acetone and/or dihydroxy acetone precursors as described in WO 01/85124 and/or erythrulose.

Kits

The disclosure also features a kit containing a lysate or total protein product described herein (e.g., a perivascular tissue lysate, an MSC total protein product (e.g., an HTPP), and/or an MSC (e.g., HUCPVC) lysate), or instructions and materials for making a lysate or total protein product (e.g., the kit may include a cryopreserved umbilical cord or cryopreserved umbilical cord vessels, or cryopreserved MSCs (e.g., HUCPVCs) and instructions for preparing a perivascular tissue lysate, an MSC total protein product (e.g., an HTPP), and/or an MSC (e.g., HUCPVC) lysate described herein). In embodiments in which the kit contains cryopreserved MSCs (e.g., HUCPVCs), the kit may contain instructions for culturing and expanding the MSCs (e.g., HUCPVCs) as an initial step in the preparation of a cultured MSC (e.g., HUCPVC) lysate or total protein product. The kit may further include reagents needed to prepare a lysate described herein, such as sterile saline (e.g., 0.9% saline), PBS, trypsin, complete medium, a cell scraper, and/or a 0.22 μM filter. In some embodiments, the lysate or total protein product is provided in a cosmetic composition described herein. A kit containing a lysate- and/or total protein product-containing cosmetic composition may further include instructions for topical application of the cosmetic composition. In some embodiments, the lysate or total protein product is provided in a dermatologic composition (e.g., the kit is obtained using a prescription). The kit containing a lysate- and/or total protein product-containing dermatologic composition can further include instructions for application (e.g., topical application) of the dermatologic composition). In some embodiments, the lysate is provided in a pharmaceutical composition (e.g., a pharmaceutical composition formulated for injection or topical administration). The kit including a lysate- and/or total protein product-containing pharmaceutical composition may further include a syringe for administration (e.g., local injection) of the pharmaceutical composition and/or instructions for administering the pharmaceutical composition to treat an autoimmune or inflammatory disease or condition, inflammation-related skin disease or condition, local inflammation, or angiogenesis-related disease or condition described herein. In some embodiments, the lysate or total protein product is provided in a cell culture reagent (e.g., a medium or medium supplement). The kit including a lysate or lysate- and/or total protein product-containing cell culture reagent may further contain instructions for application of the lysate or total protein product, or the lysate- or and/or total protein product-containing cell culture reagent to cultured cells.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of the disclosure.

### Example 1. Preparation of HUCPVC Total Protein Product (HTPP)

Protocol Overview:

Cord Dissection

The umbilical cord was disinfected with Chloraprep and then rinsed with DPBS in order to remove blood and other debris. Cord dissection was performed on a dissection table: 1) cut a shallow circumferential incision, 2) carefully and gradually peel the thin amniotic epithelial membrane. After another rinse in DPBS, the denuded cord was then separated into the three umbilical vessels (2 arteries and 1 vein) and cut into 10 cm segments.

Cryopreservation of Cord Vessels

The vessels were incubated with cryopreservation media (CryoStorCS10) on gentle rotation at 4° C. for 45 minutes. After incubation, vessels were transferred individually into cryovials containing cryopreservation media, and cryopreserved using a controlled rate freezer at 1 degree per minute freezing speed. For long term storage, the samples were stored in liquid nitrogen.

HUCPVC Isolation

A CELLstart-coated flask (1:50 dilution in PBS+/+) was preloaded with complete culture medium and placed in the incubator. The vessels were then thawed and incubated at room temperature in DPBS for 5 minutes. Another 2 rounds of incubation, 5 minutes and 10 minutes, were performed after doubling the volume. After a final rinse in DPBS, the perivascular Wharton's jelly was tentatively stripped from each vessel. The jelly was then cut into fragments approximately 2 mm by 2 mm and seeded into preloaded CELL-coated flask as P0 culture.

P0 was cultured for 7-14 days based on cell outgrowth. Upon desired confluency being reached, P0 culture was rinsed with DPBS and incubated in a recombinant cell dissociation enzyme (trypLE) at 37° C. for 10 minutes. Complete culture medium was subsequently added to the flask. The cell suspension was then filtered through a cell strainer to remove tissue debris and centrifuged at 290×g for 10 minutes at 4° C. A viable cell count was obtained by hemocytometer or ViCell technology after cells were re-suspended in complete culture medium.

HUCPVC Expansion

P1-P3 cells were then seeded at 2000 cells/cm² in human fibronectin-coated flasks (0.75 μg/cm²) preloaded with complete culture medium, 4 days per passage. At harvest, cells were dissociated with trypLE incubation at 37° C. Complete culture medium was subsequently added to the flask. The cell suspension was then centrifuged at 290×g for 10 minutes at 4° C. A viable cell count was obtained by a hemocytometer or ViCell technology after cells were re-suspended in complete culture medium. P3 cells were cryopreserved in 1:1 basal medium:EZCPZ by either a controlled rate freezer or an isopropanol-based freezing container (Mr. Frosty).

HTPP Preparation

The cells were thawed by a 2-step dilution using pre-warmed complete medium. The cell suspension was then centrifuged at 290×g for 10 minutes at 4° C. and resuspended in 0.9% saline solution. A viable cell count was obtained by a hemocytometer or ViCell technology. The cells were incubated at 37° C. in 0.9% saline solution for 6 hours with intermittent gentle mixing. The cells were homogenized 4 times for 30 seconds each time with 1-minute intervals using a bead mill homogenizer. 0.9% saline solution was then used to top up the homogenized cell mixture to 1 million cells per ml (based on the viable cell count after thawing). The final samples were filtered through 0.22 um filter and then aliquoted and stored in a −80° C. freezer.

Detailed Protocol:

1. Dilute CELLstart (CELLStart CTS—Life Technologies (A10142-01)) 1:50 with PBS (+Ca/+Mg) (Life Technologies (14040-182)), mix and add 78 μL/cm² to the tissue culture container per manufacturer instructions
2. Place in CO₂ incubator at 37° C. for 2 hours
3. Use either immediately or store for later:
a. Immediate use—aspirate coating solution and use without rinsing or
b. Later use—do not aspirate, parafilm vented caps and store at 4° C. up to 2 weeks; at time of use, aspirate coating solution and use without rinsing
   Note: In each case, after aspiration, pre-load flask with complete medium (RBI Complete medium (KT-016)) and allow to warm up in CO₂ incubator at 37° C. for at least 15 minutes prior to seeding explanted tissue or cells
   4. Open Disinfection Kit (Forceps, Mat, Chloraprep applicator) with mat laid out.
   5. Pipette 100 mL of PBS to media bottle.
   6. Using forceps, transfer umbilical cord to the mat.
   7. Place ruler over the cord to measure length and across the cord to measure the girth and record the information on the umbilical cord processing log sheet.
   8. Wipe down cord surface, including the ends, for 30 seconds with Chloraprep applicator. Allow cord to dry completely; approximately 30 seconds.
   9. Place cord in PBS rinse bottle, cap and shake vigorously to remove blood and debris. Transfer rinsed cord to a specimen container.
   8. Remove dirty mat and forceps from the BSC.
   9. Open Dissection Kit (Dissection table, Mat, Forceps (2), Scalpel handle & blade No. 15) and place table on the mat.
   10. Transfer cord to dissection table. Insert 0.5-1 cm of cord under the pressure plate and tighten all the way with screws.
   11. Make a shallow circumferential incision of the epithelium a few millimeters away from the pressure plate with the scalpel.
   12. Using forceps and blunt dissection, carefully peel the thin amniotic epithelial membrane bit by bit across the circumference at the incision site until a continuous flap of epithelium (approx. 3-4 mm) has been lifted.
   13. Holding forceps perpendicular to the cord, firmly grasp a large flap of epithelium (i.e. not grabbing by the tip) and pull it a few mm down the cord. Rotate to work all around the cord, pulling the “sock” incrementally towards the free end. Continue until the entire membrane is stripped away. Discard amniotic epithelium in a biohazard waste bin.
a. If the membrane does not separate as one whole piece and/or break apart during this step, trace back and try to re-establish the “sock”. Care must be taken as this thin layer can tear easily.
b. Be sure to remove only a thin layer. If the layer becomes too thick, use the scalpel to scrape back excess WJ.
   14. Once the entire tube of epithelium is removed, sever the cord just below the incision site.
   15. Transfer the denuded cord to the specimen container and add 50 mL of DPBS.
   16. Remove the dissection table from the BSC.
   17. Transfer cord to the mat. Using forceps, separate the 3 umbilical vessels and cut into 10 cm segments, cutting out blood clots where possible.
   18. Proceed to cryopreservation procedure, if desired. Otherwise, proceed to step 32.
   19. Place 3 vessels (2 arteries+1 vein) in a 50 mL tube and add 10 mL of CryoStor CS10 (CryoStor CS10 cryopreservation solution—BioLife Solutions (210102)). Seal cap with Parafilm.
   20. Place on a tube rotator at 4° C. for 45 minutes on gentle rotation.
   21. Transfer vessels to mat inside BSC. DO NOT discard CS10 solution.
   22. Add 1 mL of spent CS10 to a 5 mL cryovial (to prevent trapped air), then place one vessel segment inside; tap to release trapped air bubble. Top up to 4 mL with spent CS10, if space allows.
   23. Use a controlled rate freezer (CRF) with a program set at 1 deg/min freezer to cryopreserve. Once cycle is complete (>60 min), transfer samples to LN for long term storage.
   24. When ready to thaw cryopreserved vessels, pre-load the CellStart-coated T225 flask with
   24 mL of complete culture medium and place in a CO₂ incubator to warm.
   25. Remove vials (3) from LN and place in a 37° C. water bath.
   26. Thaw completely (˜2-2.5 minutes) and remove immediately while still cold.
   27. Transfer vessels to a specimen container with forceps.
   28. Add 15 mL of PBS (—Ca/—Mg) (PBS (—Ca/—Mg)—Life Technologies (14190-250)). Swirl and incubate for 5 min at RT.
   29. Add another 15 mL PBS and incubate for 5 min at RT.
   30. Add 30 mL PBS and incubate for a further 10 min at RT.
   31. Transfer vessels to a 50 mL tube and add 20 mL PBS for a final rinse. Then transfer vessel to a clean mat.
   32. Using blunt dissection, strip off the perivascular Wharton's jelly (PWJ) from each vessel, careful not to tear into the smooth muscle wall.
   33. Place PWJ in a clean 50 mL tube with the cap on to keep it from drying.
   34. Once all tissue has been stripped, pour entire PWJ content onto the cutting board and cut into small fragments using a knife. This is achieved by rocking the knife back and forth in a single plane over the tissue while applying force downward. Rotate knife perpendicular to the original plane and repeat cutting. Continue cutting in this manner until tissue fragments are approximately 2 mm×2 mm (˜5-10 minutes).
   35. Transfer minced PWJ back to 50 mL tube using a scoopula.
   36. Transfer the coated flask, now warmed, from the incubator to a biosafety cabinet (BSC).
   37. Pre-wet the tip of a wide-tip pipet (Wide-tip 10 mL pipet—VWR (89130-912)) with culture medium from the flask before using it to transfer tissue. Seed flask with all of the minced tissue.
   38. Gently tap sides and rock flask back and forth to break up tissue clumps and distribute evenly across culture surface.
   39. Return flask to incubator and culture per Explant (P0) Culture SOP below.
   40. Culture minced PWJ for 7 days undisturbed.
   41. On day 7, observe under microscope for cell outgrowth.
a. If 10 or more confluent areas are observed, proceed to step 42.
b. Otherwise, top up with 5 mL of complete medium. Monitor culture daily to decide when to harvest. If condition specified in a. is not met by day 14, discard culture.
   42. Discard spent medium and loose tissue bits using a 10 mL wide tip pipet into a waste bottle.
   43. Rinse with 24 mL PBS×2.
   Note: Do not add PBS to culture surface. Instead, add to the opposite side so that tissue bits don't get flushed off surface.
   44. Discard PBS to waste bottle.
   45. Using a regular 10 mL serological pipet, add 12 mL TrypLE (TrypLE Express—ThermoFisher (12604-021)) and incubate at 37° C. for 10 minutes.
   46. Observe. Further incubate in 5-minute intervals if needed.
   47. Tap gently to help dissociate tissue/cells.
   48. Observe under microscope to confirm all cells have dissociated. If more time is required, incubate for another 5 minutes and observe, etc.
   49. Add 24 mL of complete medium to flask.
   50. Place a cell strainer over a new 50 mL tube. Mix and transfer cell/tissue suspension to tube, straining out the tissue bits in the process.
   51. Rinse flask with 14 mL PBS and transfer to the tube by passing it through the strainer.
   52. Tilt cell strainer at an angle and tap to drain all liquid and discard strainer with tissue in a biological waster container.
   53. Centrifuge tube of cells at 290×g for 10 min/4° C.
   54. Resuspend cells in complete culture medium and count by hemocytometer or ViCell.
   55. Use 0.75 μg/cm² fibronectin (Human Fibronectin, 1 mg/mL—EMD Millipore (FC010-10MG)) and 0.133 mL of PBS (—Ca/—Mg)/cm²
   56. Mix fibronectin with PBS and use add coating volumes per PBS column to culture vessel
   57. Incubate at RT for 2 hours
   58. Aspirate coating solution and
a. Use immediately without rinsing or
b. Parafilm and store at 4° C. up to 14 days—At time of use: Pre-load flask with complete medium and allow to warm up in CO₂ incubator at 37° C. for at least 15 minutes prior to seeding cells
   59. Proceed to Cell Expansion Cultures below.
   60. Warm complete medium in 37° C. water bath for 10 minutes.
   61. Seed harvested cells at 2000 cells/cm²
   62. Culture for 4 days without feeding.
   63. Harvest at P1 and freeze down as Master Cell Bank (MCB) at 0.5-1 million cells/mL/vial or harvest at P3 as final cell product (TXP) and freeze at higher concentrations up to 15 million/mL.
a. Aspirate spent medium.
b. Add 12 mL/T225 TrypLE and incubate for 10 min at 37° C.
c. Tap gently on all sides to help detach cells.
d. Observe under microscope to confirm all cells have detached. Add extra time in increments, if required. Observe to confirm complete detachment after each incubation.
e. Add 12 mL complete medium and flush with pipette, and transfer to a 50 mL tube.
   64. Centrifuge cells at 290×g at 4° C. for 10 min.
   65. Resuspend and count on ViCell.
   66. Seed for further expansion at 2000 cells/cm² or cryopreserve, if desired.
   67. After cells have been counted, centrifuge at 290×g for 10 min at 4° C. to pellet cells.
   68. Aspirate supernatant.
   69. Flick tube to loosen pellet. Add the required volume of complete medium first, mix then dropwise add EZCPZ at a ratio of 1:1. Aliquot 1 mL/vial in 1.6 mL cryovials. Print vial labels and affix to vials.
   70. Freeze cells in a controlled rate freezer (CRF) or in an isopropanol-based freezing container (Mr. Frosty). If using a CRF, once freeze program (rate of 0.75° C./min) is completed and sample has reached −80° C., transfer to LN for long term storage. If using a Mr. Frosty, place samples in the container and place in a −80° C. freezer overnight, then transfer to LN for long term storage.
   71. Remove a 1 mL vial of cells from LN and place immediately in a 37° C. water bath.
   72. Thaw until a small sliver of ice remains (˜2 min) and transfer to a BSC.
   73. Dropwise add 1 mL of complete medium to the vial. Wait 2 min at RT.
   74. Transfer contents (2 mL) to a 15 mL tube and add 2 mL of complete medium quickly. Wait 5 min at RT.

### 75. Centrifuge at 290×g for 10 min at 4° C.

76. Resuspend in 1 mL of saline (0.9% NaCl) for counting by hemocytometer or by ViCell using 1:10 dilution.
77. Incubate at 37° C. incubator for 6 h with intermittent gentle mixing
78. Homogenize using a bead mill homogenizer for 4 rounds of 30 sec with 1 min interval on ice
79. Top up with saline to 1 million cells per mL
80. Filter homogenate through 0.22-micron syringe filter
81. Aliquot and store at −80° C.

### Example 2. Preparation of Perivascular Tissue and Stem Cell Lysates

Procedure for Preparation of Perivascular Tissue Lysate
1.1 Thaw frozen human umbilical cord vessels (one 15 mL cryopod containing 2 arteries and 1 vein for each donor lot) in 37° C. water bath for approx. 15 min
1.2 Transfer all 3 vessels to a specimen container and add 100 mL PBS (−/−) (PBS (−/−) (Life Technologies 14190)). Cap and shake to rinse out blood. Repeat rinse in a second container.
1.3 Place vessels on a silicone mat and strip the Wharton's jelly (VVJ) using forceps
1.4 Cut off and discard any blood-contaminated WJ
1.5 Cut WJ strips into small pieces (0.5-1.0 cm in length) and transfer to a specimen container
1.6 Obtain the weight of WJ tissue
1.7 Transfer WJ tissue to Baby Bullet blender cup (Baby Bullet, Capbran Holding LLC)
1.8 Rinse specimen container with 5 mL of Saline solution (0.9% saline (Baxter JB1302)) and add to blender cup
1.9 Top up with frozen saline to a final tissue weight (g): volume of saline (mL) ratio of 1:3; in case for in vitro study, use basal medium instead of saline
1.10 Use Baby Bullet blender to blend tissue continuously for 2 min
1.11 Transfer mashed tissue to 2 of 15 mL tubes
1.12 Centrifuge at 500 g for 15 min at 4° C.
1.13 Collect and pool supernatant into one tube (A)
1.14 Repeat steps 1.12 and 1.13
1.15 Centrifuge the pellets from steps 1.12 to 1.14 at 3716 g for 15 min at 4° C.
1.16 Collect supernatant into tube (A)
1.17 Repeat steps 1.15 and 1.16 until no more supernatant separation
1.18 Centrifuge the tube (A) at 500 g for 15 min at 4° C.
1.19 Sterilize supernatant from step 1.18 by filtering through 0.22-micron syringe filter
1.20 Aliquot and store at −80° C.

Procedure for Preparation of Perivascular Stem Cell Lysate
2.0 Culture and expand HUCPVCs as described in Example 1, above
2.1 A) Harvest cell expansion with trypsinization and obtain cell lysate:
   Aspirate spent culture medium
   Add pre-warmed TrypLE (TrypLE Express (Gibco 12604021)) 4 mL per 75 cm² and incubate at 37° C. for 5-10 min
   Tap gently to detach cells and observe under microscope to confirm complete cell detachment.
   Add complete medium (RBI complete medium (RoosterBio KT-016)) to culture vessel and flush cells off the culture surface.
   Collect cell suspension in a tube and perform viable cell count.
   Centrifuge at 290×g at 4° C. for 10-15 min.
   Option 1: Aspirate supernatant and perform 3 times of freeze (@˜20° C. or lower)-thaw (@RT˜37° C.) cycles; then resuspend in saline solution to 1 million cells per mL
   Option 2: Aspirate supernatant and resuspend in saline solution to 1 million cells per mL and homogenize (handheld or beads mill homogenizer) for 3 rounds of 30 sec with 1 min interval on ice
   B) Harvest cell expansion without trypsinization and obtain cell lysate:
   Aspirate spent medium
   Rinse twice with 8 mL PBS (−/−) (PBS (−/−) (Life Technologies 14190)) per 75 cm²
   Add 4 mL saline solution (0.9% saline (Baxter JB1302)) per 75 cm² and scrape cells off using cell scraper
   Observe under microscope to confirm complete cell detachment.
   Collect cell suspension in a tube
   Option 1: Perform 3 times of freeze (@−20° C. or lower)-thaw (@RT−37° C.) cycles; then top up in saline solution to 1 million cells per mL
   Option 2: Top up in saline solution to 1 million cells per mL and homogenize for 3 rounds of 30 sec with 1 min interval on ice
   C) Cryopreserve passaged cells and obtain cell lysate:
2.2 Cryopreserve passaged cells as described in Example 1.
2.3 Remove a 1 mL vial of cells from LN and place immediately in a 37° C. water bath.
2.4 Thaw until a small sliver of ice remains (˜2 min) and transfer to a BSC.
2.5 Dropwise add 1 mL of pre-warmed basal medium to the vial. Wait 2 min at RT.
2.6 Transfer contents (2 mL) to a 15 mL tube and add 2 mL of basal medium quickly. Wait 5 min at RT.
2.7 Perform viable cell count.
2.8 Centrifuge at 290×g for 10 min at 4° C.
   Option 1: Aspirate supernatant and perform 3 times of freeze (@˜20° C. or lower)-thaw (@RT˜37° C.) cycles; then resuspend in saline solution to 1 million cells per mL
   Option 2: Aspirate supernatant and resuspend in saline solution to 1 million cells per mL and homogenize for 3 rounds of 30 sec with 1 min interval on ice
2.9 Centrifuge at 290×g at 4° C. for 15 min
3.0 Filter supernatant through 0.22-micron syringe filter
3.1 Aliquot and store at −80° C.

### Example 3. Perivascular Tissue Lysate Reduces Inflammation

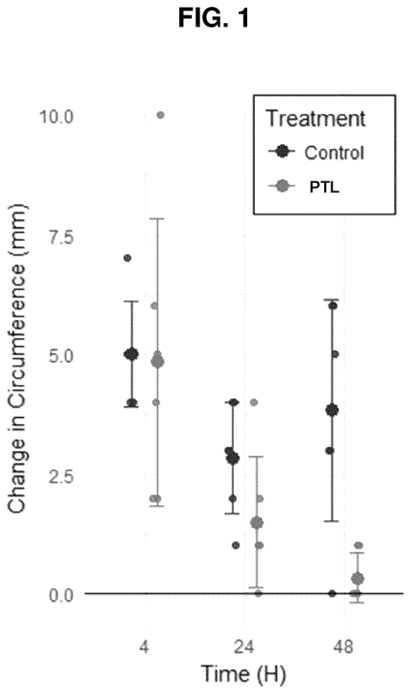

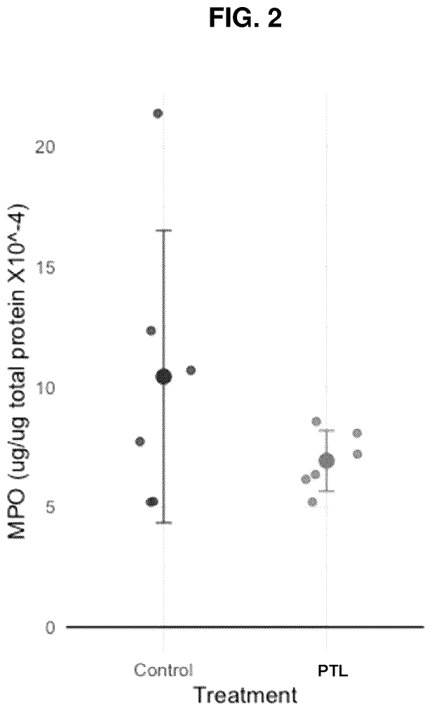

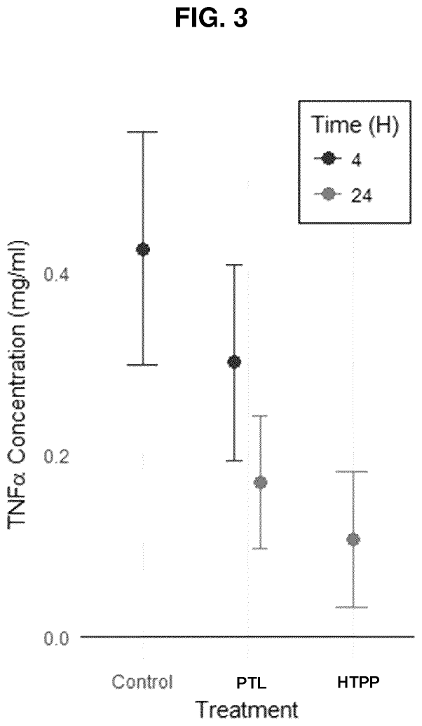

Carrageenan 1% w/v was injected into the ventral (underside) of a mouse paw at time zero and either saline (control), perivascular tissue lysate (PTL), or HTPP was injected simultaneously lateral to the Achilles tendon. Paw circumference measurements were made using a cotton thread at 4, 24, and 48 hours post-injection and normalized to the value at time zero. Carrageenan is known to have a biphasic inflammatory effect with an initial swelling at 4 hours followed by a second swelling at 72 hours. By four hours, the paws had swollen in both groups (n=6 ten-week old CD1 mice/group). As shown in FIG. 1, paw swelling had diminished in both groups by 24 hours, although it decreased more in the paws injected with PTL compared to the paws injected with saline. However, the saline group was swelling again by 48 hours while the PTL group continued to recover and paw diameter was almost back to normal. These data show that while no difference between groups was evident at 4 hours, a significant effect of PTL was seen at 48 hours (p=0.003, two-way ANOVA). Myeloperoxidase (MPO) levels were also measured by ELISA to assess the effects of saline and PTL on immune cell extravasation into the injected paw. As shown in in FIG. 2, a marked decrease in MPO level was observed in in the injected paw of mice treated with PTL compared to mice treated with saline and carrageenan. A decrease in MPO is indicative of a reduction in neutrophil infiltration. In addition, pro-inflammatory cytokine tumor necrosis factor-alpha (TNFα) concentration was also measured in the injected paws using ELISA. As shown in in FIG. 3, TNFα concentration was reduced in the injected paw of mice treated with

PTL compared to mice treated with saline (control) at four 4 hours after injection. The TNFα concentration was further reduced in the injected paw of mice treated with PTL 24 hours after injection. The concentration of TNFα in the injected paw of mice treated with HTPP was lower at 24 hours after injection than that observed in the injected paw of mice treated with PTL. These data indicate that PTL and HTPP reduce inflammation, and that HTPP may have a stronger anti-inflammatory effect than PTL.

### Example 4. Perivascular Tissue Lysate Promotes Cell Proliferation in Culture

Perivascular tissue lysate was mixed from two donors (D2 and D3) and produced by micronizing perivascular tissue (1 gram) in 3 mL of saline=100% concentration. This was then used to produce dilutions of 1%, 0.1% and 0.01% in basal medium (Rooster Bio Basal Medium). HUCPVCs were then cultured in basal medium containing the diluted perivascular tissue lysate or in basal medium alone (control).

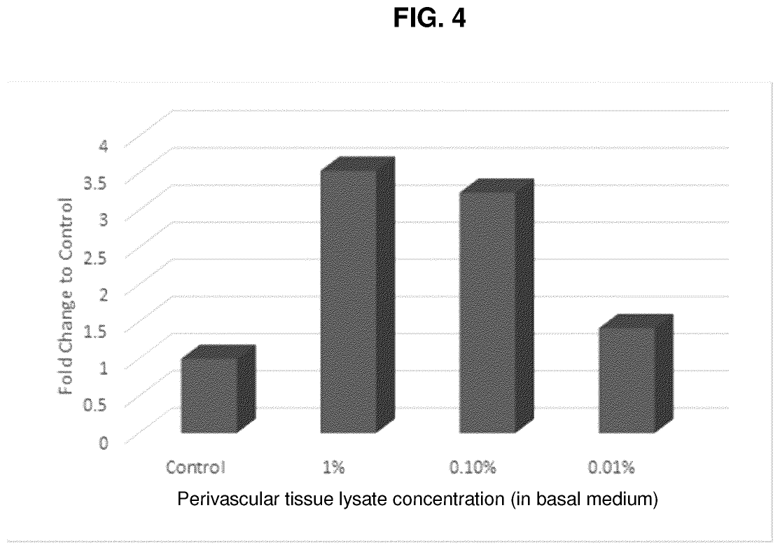

FIG. 4 contains a graph representing the number of HUCPVCs in culture on day 4 normalized to the control. As shown in FIG. 4, there were a greater number of HUCPVCs in cultures grown with basal medium containing perivascular tissue lysate compared to control.

### Example 5. HUCPVC Total Protein Product Promotes Cell Proliferation in Culture

HUCPVC Total Protein Product (HTPP) was produced from freshly-harvested HUCPVCs (1 million cells/mL saline) and lysed by repeated freeze-thaw cycles. This was then used to produce dilutions of 1%, 0.1% and 0.01% in basal medium (Rooster Bio Basal Medium). HUCPVCs were then cultured in basal medium containing the diluted HTPP or in basal medium alone (control).

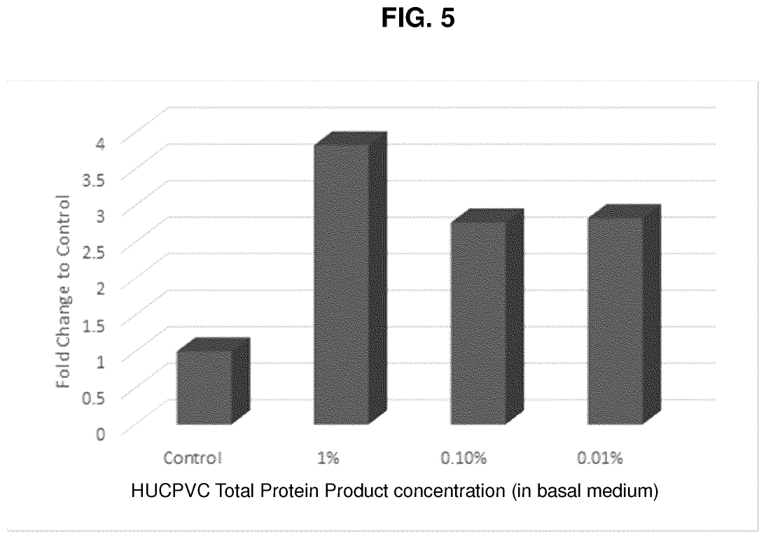

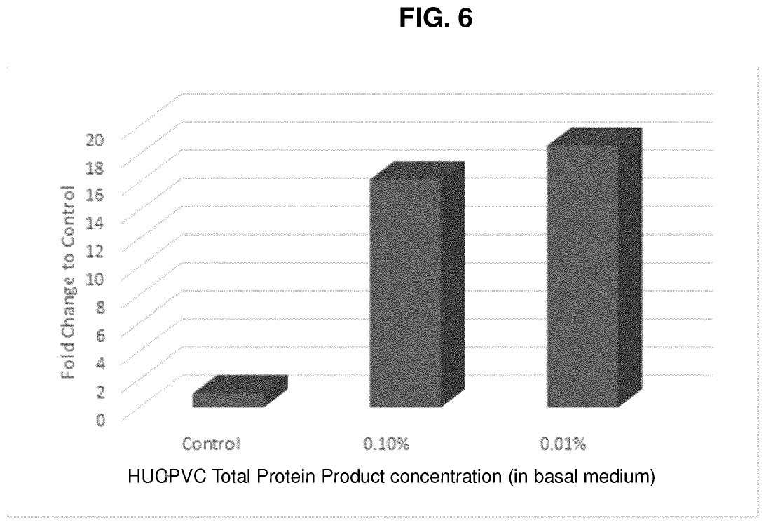

FIG. 5 contains a graph representing the number of HUCPVCs in culture on day 4 normalized to the control. As shown in FIG. 5, there were a greater number of HUCPVCs in cultures grown with basal medium containing HTPP compared to control at all dilution concentrations. FIG. 6 contains a graph representing the number of HUCPVCs in culture on day 7 normalized to the control, which demonstrates that this effect was magnified as HUCPVCs continued to divide during additional days in culture.

### Example 6. Mass Spectrometry Analysis of Samples of Perivascular Tissue and Whole Umbilical Cord Tissue

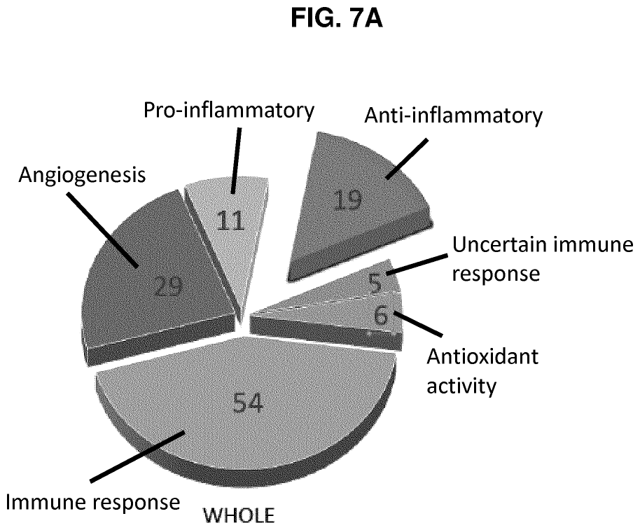

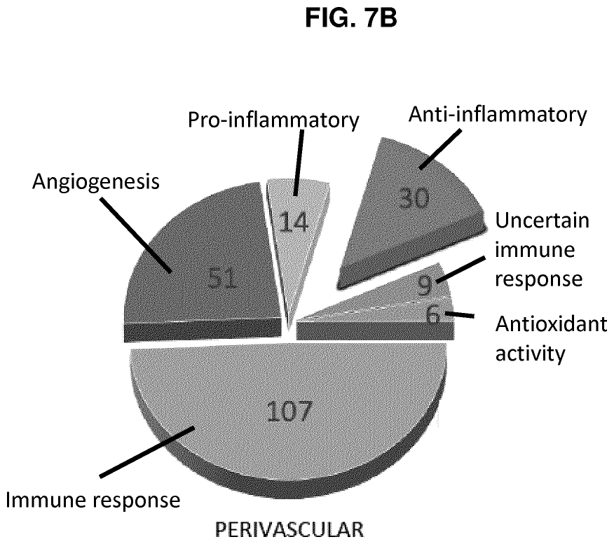

Mass spectrometry analysis was performed to identify proteins present in samples of whole cord and perivascular tissue. The proteins identified were then categorized based on functions of interest. As shown in FIGS. 5A and 5B, the total number of proteins of interest in perivascular tissue (FIG. 7B) was almost double that in whole cord tissue (FIG. 7A). Also, the percentages of the groups of proteins, while similar in both groups, show some distinct differences—particularly the immune response group.

### Example 7. The HUCPVC Total Protein Product Reduces the Number of Cells in a Joint Following Local Inflammation

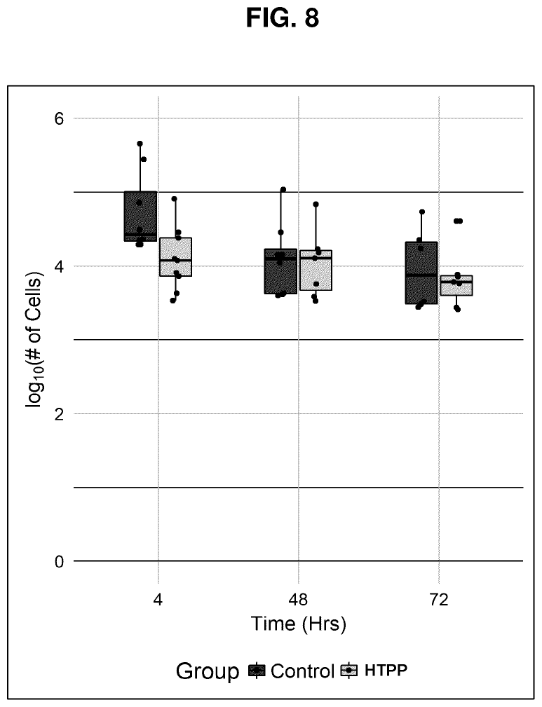

Carrageenan 1% w/v was injected into the temporomandibular (jaw) joint of a rat at time zero to create an inflammatory reaction. Saline or a 10% dilution of the HTPP (10% dilution from the production concentration based on a cell equivalent of one million cells/mL) was injected simultaneously (n=8/group). Injection volume was 0.2 microliters. Thus, the cell equivalent of 200,000 cells divided by 10=20,000 cell equivalents were injected. Synovial fluid was then aspirated from the joint at three time points: 4, 48, and 72 hours. As shown in FIG. 8, at the first time point sampled (4 hours), there was a marked decrease in the number of aspirated cells from the joint in rats administered HTPP, with the levels remaining constant after that time point. The p value for the difference at 4 hours is 0.01. These data show that the HTPP promotes a profound effect on (e.g., decreasing) the number of cells (e.g., immune cells) entering the synovial joint space as a result of the carrageenan administration. These data show that the HTPP is capable of reducing inflammation.

### Example 8. The HUCPVC Total Protein Product Contains Proteins in Membrane Bound Compartments

A bicinchoninic acid assay (BCA) was used to measure total available protein in the HTPP. This returned a value of 24 μg/mL. However, this level was increased to 44.04 μg/mL following lysis, demonstrating that approximately 50% of the protein is contained in vesicles/exosomes. The vesicles/exosomes represent material secreted by the HUCPVCs during the HTPP production protocol, demonstrating that the HTPP contains not only the intracellular and transmembrane proteins, but also proteins secreted by the cells. These data demonstrate that the HTPP contains approximately 44 micrograms of protein per million cells and that the HTPP preparation protocol generates a product containing proteins from all possible cell sources that may be more representative of the proteins produced by the cells.

### Example 9. Administration of HTPP to a Subject with Scleroderma

According to the methods disclosed herein, a physician of skill in the art can treat a patient, such as a human patient, with scleroderma to reduce joint pain, skin thickening or skin fibrosis, or skin tightness or immobility. To this end, a physician of skill in the art can administer to the human patient a HUCPVC Total Protein Product (HTPP) or a composition containing an HTPP. The HTPP may be administered to the patient, for example, by local injection to a joint or a region of skin thickening (e.g., intra-articular or subcutaneous injection) to treat joint pain or skin thickening or fibrosis associated with scleroderma, or may be applied topically to an area of thick or tightened or tightening skin. If the patient has a systemic form of scleroderma, the HTPP or HTPP-containing composition may be administered systemically, such as by intravenous injection. The HTPP or HTPP-containing composition can be administered in a therapeutically effective amount, such as from about 1,000 cell equivalents to about 1,000,000 cell equivalents (e.g., 1,000, 5,000, 10,000, 20,000, 50,000, 100,000, 250,000, 500,000, 750,000, or 1,000,000 cell equivalents). The HTPP or HTPP-containing composition can be administered bimonthly, once a month, once every two weeks, or at least once a week or more (e.g., 1, 2, 3, 4, 5, 6, or 7 times a week or more).

Following administration of the HTPP to a patient, a practitioner of skill in the art can monitor the patient's improvement in response to the therapy by a variety of methods. For example, a physician can monitor the patient's joint pain and skin thickness or tightness using standard approaches and patient self-reporting. A finding that the patient's joint pain improves, or that the patient's skin thickening or skin tightness is reduced or does not worsen, as compared to measurements taken prior to administration of the HTPP, indicates that the patient is responding favorably to the treatment. Subsequent doses can be determined and administered as needed.

### Example 10. Administration of HTPP to a Subject with Psoriasis

According to the methods disclosed herein, a physician of skill in the art can treat a patient, such as a human patient, with psoriasis to reduce inflammation, flaking, or the size or number of flaky patches on the skin, or to prevent psoriasis from worsening. To this end, a physician of skill in the art can administer to the human patient an HTPP or a composition containing an HTPP. The HTPP may be administered to the patient, for example, by local injection to or near a region of the skin exhibiting red, white, or silvery patches of skin or may be applied topically to an affected area. For topical administration, the HTPP may be formulated in an ointment or lotion. The HTPP or HTPP-containing composition can be administered in a therapeutically effective amount, such as from about 1,000 cell equivalents to about 1,000,000 cell equivalents (e.g., 1,000, 5,000, 10,000, 20,000, 50,000, 100,000, 250,000, 500,000, 750,000, or 1,000,000 cell equivalents). The HTPP or HTPP-containing composition can be administered bimonthly, once a month, once every two weeks, or at least once a week or more (e.g., 1, 2, 3, 4, 5, 6, or 7 times a week or more).

Following administration of the perivascular lysate to a patient, a practitioner of skill in the art can monitor the patient's improvement in response to the therapy by a variety of methods. For example, a physician can monitor the appearance of the patient's skin using standard approaches. A finding that the patient's psoriasis lessens (e.g., the number or size of patches of flaky, red, white, or scaly skin is reduced) or does not worsen, as compared to measurements taken prior to administration of the HTPP, indicates that the patient is responding favorably to the treatment. Subsequent doses can be determined and administered as needed.

### Example 10. Use of a Face Mask Containing an HTPP to Improve the Condition of the Skin

A subject who wishes to improve the condition, appearance, or texture of their skin can do so by applying an HTPP prepared as described herein above to their skin.

The HTPP can be applied, for example, as a topical composition, such as an ointment, lotion, cream, serum, etc. If desired, the topical composition can be applied, e.g., in connection with a mask. The mask may be a face mask (e.g., a sheet mask, cream mask, clay mask, gel mask, overnight mask (e.g., a sleeping mask), exfoliating mask, or enzyme mask) that contains the HTPP.

The subject may apply the composition to the skin (e.g., as part of a mask) 2-3 times per week, such as for at least about 15 to 30 minutes or for several hours, such as overnight.

If the mask is a sheet mask, the subject can use a fresh sheet mask for each application and discard the mask after use and may massage any remaining composition into the skin (e.g., the face, neck, and/or chest or other area of skin). If the mask is a cream, clay, gel, overnight, exfoliating, or enzyme mask, the subject may wash their skin to remove the mask after use. With continued use, the HTPP-containing mask may improve the subject's skin by reducing the appearance of redness, dryness, dark spots, or fine lines and/or improving skin brightness, firmness, texture, and hydration.

### Example 11. Administration of a Cream Containing an HTPP to a Subject with Atopic Dermatitis

According to the methods disclosed herein, a physician of skill in the art can treat a patient, such as a human patient, with atopic dermatitis (eczema). To this end, a physician of skill in the art can prescribe the patient a medicated skin cream containing an HTPP that the patient may self-administer. The subject can topically apply the HTPP-containing skin cream to the affected area to treat the atopic dermatitis and reduce redness or itching. The medicated skin cream containing the HTPP can be applied once a week, twice a week, three times a week, four times a week, five times a week, six times a week, daily, or twice a day. The medicated skin cream containing the HTPP can be applied until the atopic dermatitis clears or the redness or itching is reduced or eliminated.

Following administration of the medicated skin cream containing the HTPP by the patient, a practitioner of skill in the art can monitor the patient's improvement in response to the therapy by a variety of methods. For example, a physician can monitor the appearance of atopic dermatitis (e.g., redness) and the patient's self-reported itchiness using standard approaches. A finding that the patient's atopic dermatitis lessens or clears (e.g., redness and/or itching is reduced or eliminated) compared to measurements taken prior to application of the medicated skin cream indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of producing a human umbilical cord perivascular cell (HUCPVC) total protein product (HTPP), the method comprising:
(a) obtaining HUCPVCs;
(b) incubating the HUCPVCs in saline at about 25° C. to about 37° C. for about 15 minutes to about 24 hours;
(c) forming a homogenate from the HUCPVCs of step b); and
(d) filtering said homogenate to produce the HTPP.

2. The method of claim 1, wherein the HUCPVCs are cultured HUCPVCs.

3. The method of claim 2, wherein the method further comprises, prior to step b), passaging the HUCPVCs in culture from 2 to 10 times.

4. The method of claim 3, wherein the cultured HUCPVCs are passaged 3 times.

5. The method of any one of claims 1-4, wherein the HUCPVCs of step a) are cryopreserved HUCPVCs.

6. The method of claim 5, wherein the cryopreserved HUCPVCs were cultured prior to cryopreservation.

7. The method of claim 5 or 6, wherein the method further comprises thawing the cryopreserved HUCPVCs prior to step b).

8. The method of any one of claims 1, 3, and 4, wherein the HUCPVCs of step a) are attached to an umbilical cord blood vessel.

9. The method of claim 8, wherein the umbilical cord blood vessel is a fresh blood vessel.

10. The method of claim 8, wherein the umbilical cord blood vessel is a cryopreserved blood vessel.

11. The method of any one of claims 8-10, wherein step a) further comprises detaching the HUCPVCs from the blood vessel, and, optionally, centrifuging the detached HUCPVCs, resuspending the HUCPVCs, and counting the number of HUCPVCs prior to step b).

12. The method of any one of claims 1-11, wherein step b) comprises incubating the HUCPVCs in saline at a volume of about 500,000 cells per mL to about two million cells per mL.

13. The method of claim 12, wherein step b) comprises incubating the HUCPVCs in saline at a volume of about one million cells per mL.

14. The method of any one of claims 1-13, wherein step b) comprises incubating the HUCPVCs in saline for about 6 hours.

15. The method of any one of claims 1-14, wherein step b) comprises incubating the HUCPVCs at about 37° C.

16. The method of any one of claims 1-15, wherein the method comprises adding saline to the homogenate formed in step c) to reach a concentration of about one million cells per mL.

17. The method of any one of claims 1-16, wherein the filtering is performed using a filter of about 0.2 μM to about 0.45 μM.

18. The method of claim 17, wherein the filtering is performed using a filter of about 0.22 μM.

19. The method of any one of claims 1-18, wherein the method further comprises aliquoting the HTPP after filtration.

20. The method of any one of claims 1-19, wherein the method further comprises storing the HTPP at about −80° C. or lower until use.

21. The method of claim 20, wherein the aliquot comprises an amount of protein corresponding to a cell equivalent of at least about one million cells.

22. The method of claim 21, wherein the volume of the aliquot is about one milliliter (mL) or less.

23. The method of any one of claims 1-22, wherein the homogenate is formed using a bead mill homogenizer.

24. The method of any one of claims 1-23, wherein the HTPP comprises an amount of protein corresponding to a cell equivalent of about one million cells per mL.

25. The method of any one of claims 1-24, wherein the amount of protein in the HTPP is about 20 μM to about 80 μM per mL.

26. The method of claim 25, wherein the amount of protein in the HTPP is about 45 μM per mL.

27. A composition comprising the HTPP produced according to the method of any one of claims 1-26.

28. The composition of claim 27, further comprising a pharmaceutically acceptable excipient.

29. A method of treating a subject having an autoimmune or inflammatory disease or condition comprising administering to the subject a therapeutically effective amount of the composition of claim 27 or 28.

30. The method of claim 29, wherein the autoimmune or inflammatory disease or condition is selected from the group consisting of achalasia, acne vulgaris, acute disseminated encephalomyelitis (ADEM), acute necrotizing hemorrhagic leukoencephalitis, Addison's disease, adjuvant-induced arthritis, adult Still's disease, agammaglobulinemia, alopecia areata, amyloidosis, ankylosing spondylitis, anti-GBM/anti-TBM nephritis, antiphospholipid syndrome (APS), atopic dermatitis, autoimmune angioedema, autoimmune aplastic anemia, autoimmune dysautonomia, autoimmune encephalomyelitis, autoimmune gastric atrophy, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune orchitis, autoimmune pancreatitis, autoimmune retinopathy, autoimmune thrombocytopenic purpura (ATP), autoimmune thyroid disease, autoimmune urticaria, axonal & neuronal neuropathy (AMAN), Balo disease, Behcet's disease, benign mucosal pemphigoid, bullous pemphigoid, Castleman disease, celiac disease, Chagas disease, chronic inflammatory demyelinating polyneuropathy (CIDP), chronic recurrent multifocal osteomyelitis (CRMO), Churg-Strauss syndrome, cicatricial pemphigoid, Crohn's disease, Cogan's syndrome, collagen-induced arthritis, cold agglutinin disease, congenital heart block, coxsackie myocarditis, CREST syndrome, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, epidermolysis bullosa, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalopathy, Hashimoto's thyroiditis, hemolytic anemia, Henoch-Schonlein purpura (HSP), herpes gestationis, Hidradenitis Suppurativa (Acne Inverse), hypogammaglobulinemia, idiopathic thrombocytopenic purpura (ITP), IgA nephropathy, IgG4-related sclerosing disease, inclusion body myositis, interstitial cystitis, inflammatory bowel disease, juvenile arthritis, juvenile diabetes (type 1 diabetes), juvenile myositis, Kawasaki disease, Lambert-Eaton syndrome, leukocytoclastic vasculitis, lichen planus, lichen sclerosus, ligneous conjunctivitis, linear IgA disease (LAD), lupus (Systemic Lupus Erythematosus), Lyme disease chronic, Meniere's disease, microscopic polyangiitis, mixed connective tissue disease (MCTD), Mooren's ulcer, Mucha-Habermann disease, multifocal motor neuropathy (MMN), multiple sclerosis, myasthenia gravis, myositis, narcolepsy, neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, pars planitis (peripheral uveitis), pemphigus, peripheral neuropathy, perivenous encephalomyelitis, pernicious anemia, POEMS syndrome, polyarteritis nodosa, polyglandular syndromes type I, II, III, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, pyoderma gangrenosum, pure red cell aplasia, Raynaud's phenomenon, reactive arthritis, reflex sympathetic dystrophy, relapsing polychondritis, restless legs syndrome, retroperitoneal fibrosis, rheumatic fever, rheumatoid arthritis, rosacea, sarcoidosis, Schmidt syndrome, scleritis, scleroderma, primary sclerosing cholangitis, chronic sclerosing sialadenitis, Sjogren's syndrome, sperm & testicular autoimmunity, stiff person syndrome, subacute bacterial endocarditis (SBE), Susac's syndrome, Sweet syndrome, sympathetic ophthalmia, Takayasu's arteritis, thrombocytopenic purpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, vitiligo, and Vogt-Koyanagi-Harada Disease.

31. The method of claim 30, wherein the autoimmune or inflammatory disease or condition is scleroderma.

32. The method of claim 30, wherein the autoimmune or inflammatory disease or condition is rheumatoid arthritis.

33. The method of claim 30, wherein the autoimmune or inflammatory disease or condition is Sjogren's syndrome.

34. A method of treating a subject having an autoimmune or inflammation-related skin disease or condition comprising administering to the subject a therapeutically effective amount of the composition of claim 27 or 28.

35. The method of claim 34, wherein the autoimmune or inflammation-related skin disease condition is atopic dermatitis, psoriasis, scleroderma, dermatomyositis, epidermolysis bullosa, pemphigus, bullous pemphigoid, alopecia areata, ocular cicatricial pemphigoid, dermatitis herpetiformis, linear IgA disease, dermatomyositis, lupus, vasculitis, Bechet's disease, lichen planus, rosacea, acne vulgaris, pyoderma gangrenosum, Hidradenitis Suppurativa, Sweet syndrome, autoimmune urticaria, a rash, or itchy skin.

36. A method of improving cosmetic appearance in a subject in need thereof comprising administering to the subject a composition of claim 27 or 28.

37. The method of claim 36, wherein the method comprises reducing skin redness, reducing skin dullness, reducing skin dryness, reducing the appearance of fine lines and wrinkles, reducing the appearance of dark spots, reducing hyperpigmentation, reducing uneven skin texture, reducing acne, reducing the appearance of a scar or pockmark, reducing the appearance of a stretch mark, reducing pore size, firming the skin, brightening the skin, hydrating the skin, reducing skin irritation, soothing sunburned skin, increasing the production of collagen, and/or reducing collagen degradation.

38. A method of reducing local inflammation in a subject in need thereof, the method comprising administering to the subject an effective amount of the composition of claim 27 or 28.

39. The method of claim 38, wherein the subject has inflammation of a bursa, inflammation of a tendon, inflammation of a joint, inflammation of the jaw, inflammation of a gland, back pain, neck pain, sciatica, trigger finger, carpal tunnel syndrome or another entrapment syndrome, synovitis, rotator cuff syndrome, impingement syndrome, alopecia, frozen shoulder syndrome, fasciitis, or gout.

40. The method of claim 39, wherein inflammation of the joint is associated with arthritis.

41. A method of increasing or inducing angiogenesis in a subject in need thereof comprising administering to the subject an effective amount of the composition of claim 27 or 28.

42. The method of claim 41, wherein the subject has a cardiovascular disease, a wound, abnormal vasculature, poor vascularization, has received a tissue transplant, or is at risk of losing a limb.

43. The method of any one of claims 29-42, wherein the composition is administered by injection.

44. The method of any one of claims 29-42, wherein the composition is administered topically.

45. The method of any one of claims 29-42, wherein the composition is administered by inhalation.

46. The method of any one of claims 29-45, wherein the composition is administered locally to a site of inflammation.

47. A method of improving the health, proliferation, and/or longevity of cultured HUCPVCs, the method comprising contacting the cultured cells with an effective amount the HTPP produced according to the method of any one of claims 1-26 or the composition of claim 27.

48. A cosmetic composition comprising the HTPP produced according to the method of any one of claims 1-26.

49. The composition of claim 48, wherein the cosmetic composition is formulated as a face or body cream, face or body lotion, ointment, oil, serum, essence, gel, mist, mask, foundation, blush, eyeshadow, mascara, eyeliner, lip product, setting powder, setting spray, tinted moisturizer, BB cream, CC cream, primer, tinted under-eye cream, concealer, nail product, light-protective product, after sun product, skin cleansing product, bath product, skin-tanning product, deodorant, antiperspirant, hair removal product, shaving product, fragrance, insect repellant, or hair care product.

50. The composition of claim 48 or 49, wherein the cosmetic composition further comprises a cosmetic active ingredient.

51. The composition of claim 50, wherein the cosmetic active ingredient is tocopherol, tocopherol acetate, tocopherol palmitate, deoxyribonucleic acid, retinol, bisabolol, allantoin, phytantriol, panthenol, an amino acid, an essential oil, a plant extract, a vitamin complex, a retinoid, Vitamin C, Vitamin A, an alpha-hydroxy acid, a beta-hydroxy acid, a glycolic acid, a kojic acid, an ascorbic acid, a hyaluronic acid, alpha-lipolic acid, hydroquinone, copper peptide, Vitamin E, dimethylaminoethanol (DMAE), niacinamide, a ceramide, a pseudoceramide, a curcuminoid, or an antioxidant.

52. The composition of any one of claims 48-51, wherein the cosmetic composition further comprises a fatty alcohol, fatty acid ester, natural or synthetic triglyceride, pearlescent wax, hydrocarbon oil, silicone or siloxane, fluorinated or perfluorinated oil, emulsifier, superfatting agent, surfactant, consistency regulator/thickener, theology modifier, polymer, deodorizing active ingredient, anti-dandruff agent, film former, hydrotropic agent, preservative, bacteria inhibiting agent, perfume oil, colorant, polymeric bead, hollow sphere, solubilizer, structurant, opacifier, complexing agent, or insect repellant.

53. The composition of any one of claims 48-52, wherein the cosmetic composition is formulated for topical administration.

54. A dermatologic composition comprising the HTPP produced according to the method of any one of claims 1-26.

55. The composition of claim 54, wherein the cosmetic composition is formulated as a face or body cream, lotion, ointment, gel, or spray.

56. The composition of claim 54 or 55, wherein the cosmetic composition further comprises a dermatologic active ingredient.

57. The composition of claim 56, wherein the dermatologic active ingredient is a steroid, a corticosteroid, coal tar, a retinoid, a calcineurin inhibitor, a vitamin D analog, doxepin, benzoyl peroxide, azeliac acid, dapsone, a phosphodiesterase-4 inhibitor, anthralin, an anti-bacterial agent, or an anti-fungal agent.

58. The composition of any one of claims 54-57, wherein the dermatologic composition is formulated for topical administration.

59. A method of making a perivascular tissue lysate, comprising:
(a) obtaining one or more umbilical cord vessels;
(b) removing Wharton's Jelly adjacent to the umbilical cord vessels;
(c) homogenizing the Wharton's Jelly to produce a homogenate;
(d) centrifuging the homogenate to produce a supernatant and a pellet;
(e) collecting the supernatant in a collection tube;
(f) repeating steps (d) and (e) using the same collection tube;
(g) centrifuging the collection tube to produce a final supernatant; and
(h) sterilizing the final supernatant to produce the perivascular tissue lysate.

60. The method of claim 59, wherein the one or more umbilical cord vessels are rinsed to remove blood prior to step (b).

61. The method of claim 60, wherein the one or more umbilical cord vessels are rinsed with phosphate buffered saline.

62. The method of any one of claims 59-61, wherein blood-contaminated Wharton's Jelly is discarded prior to step (c).

63. The method of any one of claims 59-62, wherein the Wharton's Jelly is weighed prior to step (c) and the method further comprises adding saline or basal medium to the Wharton's Jelly to a final tissue weight (g):volume of saline (mL) ratio of 1:3.

64. The method of any one of claims 59-63, wherein the final supernatant is sterilized using a 0.22 μM filter.

65. The method of any one of claims 59-64, wherein the one or more umbilical cord vessels is freshly isolated from an umbilical cord.

66. The method of any one of claims 59-64, wherein the one or more umbilical cord vessels is frozen.

67. The method of claim 66, wherein the method further comprises a step of thawing the frozen umbilical cord vessels prior to step (b).

68. The method of claim 67, wherein the frozen umbilical cord vessels are thawed in a water bath at about 37° C.

69. The method of any one of claims 59-68, wherein the centrifuging of step (d) and step (g) is performed at 500×g for about 15 minutes at about 4° C.

70. The method of any one of claims 59-69, wherein the method further comprises aliquoting the perivascular tissue lysate after step (h).

71. The method of any one of claims 59-70, wherein the method further comprises storing the perivascular tissue lysate at −80° C. or lower until use.

72. The method of any one of claims 59-71, wherein the one or more umbilical cord vessels comprise two arteries and one vein.

73. A method of making a mesenchymal stem cell (MSC) lysate; comprising (a) obtaining cultured MSCs grown on a surface of a vessel;
(b) aspirating a cell culture medium from the vessel;
(c) contacting the cultured MSCs with trypsin until the MSCs detach from the surface of the vessel;
(d) flushing the MSCs from the surface of the vessel using a complete medium;
(e) collecting the complete medium containing the MSCs in a tube;
(f) centrifuging the tube containing the MSCs;
(g) aspirating the supernatant, and
(i) performing three freeze-thaw cycles prior to resuspending the MSCs to one million cells per mL; or
(ii) resuspending the MSCs in saline solution to one million cells per mL prior to performing three rounds of homogenization on ice;
(h) centrifuging the solution containing the MSCs; and
(i) collecting and sterilizing the supernatant to produce the MSC lysate.

74. A method of making a mesenchymal stem cell (MSC) lysate; comprising
(a) obtaining cultured MSCs grown on a surface of a vessel;
(b) aspirating a cell culture medium from the vessel;
(c) adding saline to the vessel and scraping the MSCs off of the surface using a cell scraper;
(d) collecting the saline containing the MSCs in a tube;
(e) centrifuging the tube containing the MSCs;
(f) aspirating the supernatant, and
(i) performing three freeze-thaw cycles prior to resuspending the MSCs to one million cells per mL; or
(ii) resuspending the MSCs in saline solution to one million cells per mL prior to performing three rounds of homogenization on ice;
(g) centrifuging the solution containing the MSCs; and
(h) collecting and sterilizing the supernatant to produce the MSC lysate.

75. A method of making a mesenchymal stem cell (MSC) lysate, comprising:
(a) obtaining cryopreserved MSCs;
(b) thawing the MSCs;
(c) adding basal medium to the MSCs to achieve a total volume of 4 mL in a tube;
(d) centrifuging the tube containing the MSCs;
(e) aspirating the supernatant, and
(i) performing three freeze-thaw cycles prior to resuspending the MSCs to one million cells per mL; or
(ii) resuspending the MSCs in saline solution to one million cells per mL prior to performing three rounds of homogenization on ice;
(g) centrifuging the solution containing the MSCs; and
(h) collecting and sterilizing the supernatant to produce the MSC lysate.

76. The method of any one of claims 73-75, wherein the supernatant is sterilized using a 0.22 μM filter.

77. The method of any one of claims 73-76, wherein a cell count is performed prior to centrifuging the tube containing the MSCs.

78. The method of any one of claims 73-77, wherein the centrifuging of the tube or the solution containing the MSCs is performed at 290×g for about 10 minutes at about 4° C.

79. The method of any one of claims 73-78, wherein the freeze-thaw cycles of step (i) are performed by freezing the cells at −20° C. or lower and thawing the cells at about 25° C. to about 37° C.

80. The method of any one of claims 73-79, wherein the method further comprises aliquoting the MSC lysate after sterilization.

81. The method of any one of claims 73-80, wherein the method further comprises storing the MSC lysate at −80° C. or lower until use.

82. The method of any one of claims 73-81, wherein the MSC is a human umbilical cord perivascular cell (HUCPVC).

83. The method of any one of claims 73-81, wherein the MSC is an MSC isolated from bone marrow, adipose tissue, amniotic fluid, amniotic membrane, dental tissue, endometrium, limb bud, menstrual blood, peripheral blood, umbilical cord blood, placenta, fetal membrane, embryonic yolk sac, salivary gland, skin, foreskin, synovial fluid, or sub-amniotic umbilical cord lining membrane.

84. The lysate made by the method of any one of claims 59-83.

85. A kit comprising the HTPP produced according to method of any one of claims 1-26, the composition of claim 27 or 28, the cosmetic composition of any one of claims 48-53, the dermatologic composition of any one of claims 54-58, or materials for producing the HTPP, composition, cosmetic composition, or dermatologic composition.

86. The kit of claim 85, wherein the materials comprise isolated umbilical cord vessels, isolated perivascular tissue, or cultured HUCPVCs and instructions for producing a HTPP.

87. A method of producing an HTPP, the method comprising:
(a) obtaining HUCPVCs;
(b) homogenizing the HUCPVCs, optionally in saline, wherein the homogenate comprises exosomes and/or vesicles from the HUCPVCs; and
(c) filtering said homogenate to produce the HTPP.

88. The method of claim 87, wherein the HTPP comprises about 200,000 to about 5000 million HUCPVC equivalents.

89. The method of claim 88, wherein the HTPP comprises a volume of from about 0.01 mL to about 1 L.

90. A composition produced by the method of any one of claims 87-89.

91. The composition of claim 90, wherein the composition comprises from about 200,000 to about 1 million HUCPVC equivalents in a volume of about 1 mL or less.

92. A method of treating a disease in a subject comprising administering the composition of claim 90 or 91 to the subject.
